(19) United States
(12) Patent Application Publication
(10) Pub. No.: US 2023/0062486 A1
(43) Pub. Date: Mar. 2, 2023

(54) KRAS G12C INHIBITOR AND PHARMACEUTICAL USE THEREOF

(71) Applicant: Betta Pharmaceuticals Co., LTD, Hangzhou (CN)

(72) Inventors: Hao Wu, Hangzhou (CN); Xiaoping Chen, Hangzhou (CN); Yuan Lu, Hangzhou (CN); Jun Yu, Hangzhou (CN); Xiujun Xie, Hangzhou (CN); Jiangqi He, Hangzhou (CN); Shuibiao Fu, Hangzhou (CN); Qi Shen, Hangzhou (CN); Letian Zhang, Hangzhou (CN); Xiaoguan Zhu, Hangzhou (CN); Hong Lan, Beijing (CN); Jiabing Wang, Beijing (CN); Lieming Ding, Hangzhou (CN)

(21) Appl. No.: 17/757,688

(22) PCT Filed: Dec. 18, 2020
(86) PCT No.: PCT/CN2020/137538

§ 371 Date: Jun. 17, 2022

(30) Foreign Application Priority Data
Dec. 19, 2019 (CN) PCT/CN2019/126655

(51) Int. Cl. C07D 487/04; A61K 31/4985; A61P 35/00

(52) U.S. Cl. CPC C07D 487/04; A61K 31/4985; A61P 35/00

(57) ABSTRACT

Provided are compounds of formula (I), which have KRAS mutation tumor regulating activity. Also provided are a method for providing these compounds and a pharmaceutical composition comprising the same.

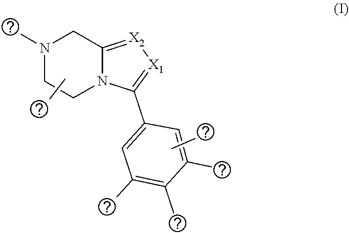

### TECHNICAL FIELD

The present invention relates to a novel murine sarcoma virus oncogene (KRAS) inhibitor and a pharmaceutically acceptable salt thereof, which can be used for regulating the activity of G12C mutant KRAS protein and treating proliferative diseases such as cancer. The present invention also relates to methods for the preparation of these compounds and pharmaceutical compositions containing them.

### BACKGROUND TECHNIQUE

Clinical data shows that RAS is the gene with the highest mutation rate in human tumors, about 20-30% of all tumors have RAS mutations, about 98% of pancreatic cancer, 52% of colon cancer, 43% of multiple myeloma, and 32% of lung adenocarcinomas have RAS gene mutations. The most common mutation of RAS is point mutation, which often occurs at codons 12, 13, and 61, of which the 12th codon mutation is the most common. KRAS-G12C mutations account for approximately 10-20% of KRAS mutations and 14% in non-small cell lung cancer. However, drug formulation is difficult because KRAS mutant proteins lack a drug-binding pocket.

RAS protein is a low-molecular-weight guanosine triphosphate (GTP) binding protein with only one polypeptide chain. It includes two conformations: an active GTP binding conformation and an inactive GDP binding conformation. Under certain conditions, they can be transformed into each other to form the RAS cycle and regulate the activation of multiple downstream signaling pathways. The most important ones include the RAF-MEK-ERK and PI3K-AKT-mTOR signaling pathways. RAS is known as “molecular switch” of the transmission of cellular signaling network. Under normal circumstances, RAS is in an inactive state combined with GDP, and RAS is activated after receiving upstream signal stimulation, and the signal chain is only temporarily active. However, when RAS is mutated, the frequency of exchange between RAS and GDP/GTP is accelerated. RAS can bind to GTP for a long time, so that RAS and downstream signals are activated for a long time, and cell proliferation is out of control, leading to malignant transformation of cells.

The RAS gene family contains three functional genes, namely HRAS, NRAS, and KRAS, which are located on chromosomes 11, 12, and 1, respectively. Because the binding ability of GTP to RAS is very strong at the level of picomole, it is difficult to find small molecules that can competitively inhibit their binding, and the surface of RAS protein is too smooth and lacks structural space for small molecules or drugs to bind. For more than 30 years, there have been no breakthroughs in the search for drugs that specifically target this small GTPase. Therefore, KRAS is generally regarded as an “Undruggable Target” protein target. At present, most KRAS-mutated tumors rely on chemotherapy alone, and there is no targeted drug or targeted therapy with relatively good efficacy.

### SUMMARY

The present invention provides a compound represented by general formula (I), or a tautomer, pharmaceutically acceptable salt, solvate, chelate, non-covalent complex or prodrug thereof,

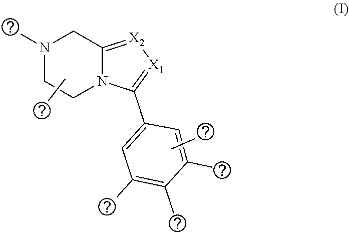

wherein,

R¹ or R³ is independently selected from the group consisting of H, amino, cyano, halogen, hydroxy, substituted or unsubstituted C₁₋₈ alkyl, substituted or unsubstituted C₃₋₈ cycloalkyl, substituted or unsubstituted C₂₋₈ alkene, and substituted or unsubstituted C₁₋₃ alkoxy;

R² is selected from the group consisting of substituted or unsubstituted C₃₋₁₀ cycloalkyl, substituted or unsubstituted C₃₋₁₀ heterocyclyl, substituted or unsubstituted C₆₋₁₂ aryl and substituted or unsubstituted C₅₋₁₂ heteroaryl;

R⁴ is selected from the group consisting of H, amino, cyano, halogen, hydroxy, substituted or unsubstituted C₁₋₈ alkyl, substituted or unsubstituted C₂₋₈ alkenyl, substituted or unsubstituted C₃₋₁₀ cycloalkyl, substituted or unsubstituted C₃₋₁₀ heterocyclyl, substituted or unsubstituted C₆₋₁₂ aryl, and substituted or unsubstituted C₅₋₁₂ heteroaryl; or

R⁴ and R¹ or together with R³ together with the atom to which it is attached form substituted or unsubstituted C₃₋₁₀ cycloalkyl, substituted or unsubstituted C₃₋₁₀ heterocyclyl, substituted or unsubstituted C₆₋₁₀ aryl, or substituted or unsubstituted C₅₋₁₂ heteroaryl;

X₁ is N or CR⁵, wherein, R⁵ is selected from the group consisting of H, amino, cyano, halogen, hydroxyl, substituted or unsubstituted C₁₋₈ alkyl, substituted or unsubstituted C₂₋₈ alkenyl, substituted or unsubstituted C₃₋₁₀ cycloalkyl, substituted or unsubstituted C₃₋₁₀ heterocyclyl, substituted or unsubstituted C₆₋₁₂ aryl, and substituted or unsubstituted C₅₋₁₂ heteroaryl;

X₂ is N or CR⁶, wherein, R⁶ is selected from the group consisting of H, amino, cyano, halogen, hydroxyl, substituted or unsubstituted C₁₋₈ alkyl, substituted or unsubstituted C₂₋₈ alkenyl, substituted or unsubstituted amido, substituted or unsubstituted aminopyridyl, and substituted or unsubstituted pyrrolidinyloxy;

R⁷ is substituted or unsubstituted acryloyl;

R⁸ is selected from the group consisting of H, C₁₋₈ alkyl, C₁₋₈ alkoxy and C₁₋₈ haloalkyl;

m or n are independently selected from the group consisting of 0, 1 and 2;

wherein any of heterocyclyl or heteroaryl optionally contains 1, 2 or 3 heteroatoms independently selected from the group consisting of N, O and S.

In one aspect, R¹ or R³ in formula (I) is independently selected from the group consisting of hydroxy, halogen, C₂₋₃ alkenyl, C₂₋₃ alkyl, cyclopropyl, C₁₋₃ alkoxy and haloalkyl substituted C₁₋₃ alkoxy.

In one aspect, R¹ or R³ in formula (I) is independently selected from the group consisting of halogen, C₂₋₃ alkenyl and —O(C₁₋₂ alkylene)CF₃.

In one aspect, R¹ or R³ in formula (I) is independently selected from the group consisting of F, Cl,

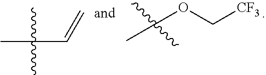

In one aspect, R² in formula (I) is selected from the group consisting of C₃₋₁₀ cycloalkyl, C₃₋₁₀ heterocyclyl, C₆₋₁₂ aryl and C₅₋₁₂ heteroaryl, wherein any of the C₃₋₁₀ cycloalkyl, C₃₋₁₀ heterocyclyl, C₆₋₁₂ aryl or C₅₋₁₂ heteroaryl is unsubstituted or optionally substituted with halogen, hydroxy, amino or C₁₋₆ alkyl.

In one aspect, R² in formula (I) is

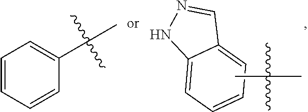

wherein

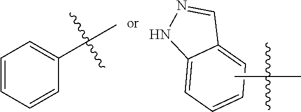

is optionally substituted by halogen, hydroxyl or amino.

In one aspect, R² in formula (I) is

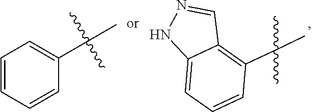 [SMILES: *c1ccccc1 ; *c1cccc2[nH]ncc12]

wherein

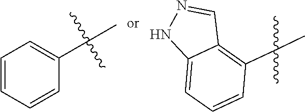 [SMILES: *c1ccccc1 ; *c1cccc2[nH]ncc12]

is optionally substituted by halogen, hydroxyl or amino.

In one aspect, R² in formula (I) is selected from the group consisting of

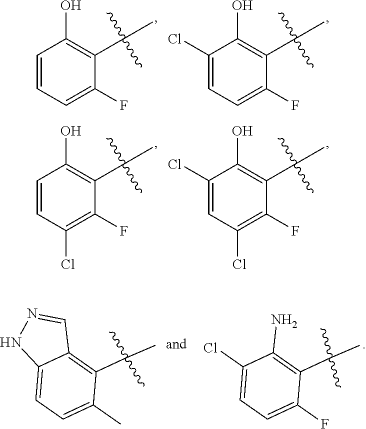

In one aspect, R⁴ in formula (I) is selected from the group consisting of H, halogen and C₁₋₃ alkyl.

In one aspect, R⁴ in formula (I) is H.

In one aspect, X₁ in formula (I) is N or CR⁵, wherein, R⁵ is selected from the group consisting of H, halogenated C₁₋₃ alkyl and

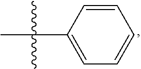 [SMILES: *c1ccccc1]

wherein

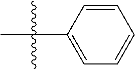 [SMILES: *c1ccccc1]

is unsubstituted or substituted by C₁₋₃ alkyl.

In one aspect, X₁ in formula (I) is N or CR⁵, wherein, R⁵ is selected from the group consisting of H, —CF₃ and

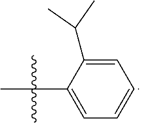 [SMILES: *c1ccccc1C(C)C]

In one aspect, X₂ in formula (I) is N or CR⁶, wherein R⁶ is H.

In one aspect, R⁷ in formula (I) is halo or unsubstituted acryloyl.

In one aspect, R⁷ in formula (I) is

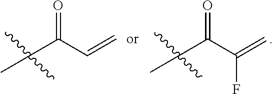

In one aspect, R⁸ in formula (I) is selected from the group consisting of H, halogen and C₁₋₃ alkyl.

In one aspect, R⁸ in formula (I) is H.

The present invention further provides a compound, or tautomer or pharmaceutically acceptable salt, wherein the compound is selected from the group consisting of:
1)1-(3-(2-Chloro-2′,6-difluoro-6′-hydroxy-[1,1′-biphenyl]-4-yl)-5,6-dihydro-[1,2,4]triazolo [4,3-a]pyrazin-7(8H)-yl)prop-2-en-1-one;
2)1-(3-(2,3′-Dichloro-6,6′-difluoro-2′-hydroxy-[1,1′-biphenyl]-4-yl)-5,6-dihydro-[1,2,4]triazolo[4,3-a]pyrazin-7(8H)-yl)prop-2-en-1-one; or
1-(3-(2,3′-Dichloro-2′,6-difluoro-6′-hydroxy-[1,1′-biphenyl]-4-yl)-5,6-dihydro-[1,2,4]triazolo[4,3-a]pyrazin-7(8H)-yl)prop-2-en-1-one;
3)1-(3-(2,3′,5′-Trichloro-2′,6-difluoro-6′-hydroxy-[1,1′-biphenyl]-4-yl)-5,6-dihydro-[1,2,4]Triazolo[4,3-a]pyrazin-7(8H)-yl)prop-2-en-1-one;
4)1-(3-(2-chloro-2′,6-difluoro-6′-hydroxy-[1,1′-biphenyl]-4-yl)-5,6-dihydro-[1,2,4]triazolo [4,3-a]pyrazin-7(8H)-yl)-2-fluoroprop-2-en-1-one;
5)1-(3-(3-chloro-5-fluoro-4-(5-methyl-1H-indazol-4-yl)phenyl)-5,6-dihydro-[1,2,4]triazolo [4,3-a]pyrazin-7(8H)-yl)prop-2-en-1-one;
6)1-(3-(2-Chloro-2′,6-difluoro-6′-hydroxy-[1,1′-biphenyl]-4-yl)-5,6-dihydroimidazo[1,2-a]pyrazin-7(8H)-yl)prop-2-en-1-one;
7)1-(3-(2′-Amino-2,3′-dichloro-6,6′-difluoro-[1,1′-biphenyl]-4-yl)-5,6-dihydro-[1,2,4]triazolo[4,3-a]pyrazin-7(8H)-yl)prop-2-en-1-one;
8)1-(3-(2-Chloro-2′,6-difluoro-6′-hydroxy-[1,1′-biphenyl]-4-yl)-5,6-dihydroimidazo[1,5-a]pyrazin-7(8H)-yl)prop-2-en-1-one;
9)1-(3-(2,3′-Dichloro-6,6′-difluoro-2′-hydroxy-[1,1′-biphenyl]-4-yl)-5,6-dihydroimidazo [1,5-a]pyrazin-7(8H)-yl)prop-2-en-1-one; or
1-(3-(2,3′-Dichloro-2′,6-difluoro-6′-hydroxy-[1,1′-biphenyl]-4-yl)-5,6-dihydroimidazo[1,5-a]pyrazin-7(8H)-yl)prop-2-en-1-one;
10)1-(3-(2,3′,5′-Trichloro-2′,6-difluoro-6′-hydroxy-[1,1′-biphenyl]-4-yl)-5,6-dihydroimidazo[1,5-a]pyrazin-7(8H)-yl)prop-2-en-1-one;
11)1-(3-(2,2′-Difluoro-6′-hydroxy-6-vinyl-[1,1′-biphenyl]-4-yl)-5,6-dihydro-[1,2,4] triazolo [4,3-a]pyrazin-7(8H)-yl)prop-2-en-1-one;
12)1-(3-(2-Chloro-2′,6-difluoro-6′-hydroxy-[1,1′-biphenyl]-4-yl)-2-(2-isopropylphenyl)-5,6-dihydroimidazo[1,2-a]pyrazin-7(8H)-yl)prop-2-en-1-one;
13) 1-(3-(2,3′-dichloro-6,6′-difluoro-2′-hydroxy-[1,1′-biphenyl]-4-yl)-2-(2-isopropylphenyl)-5,6-dihydroimidazo[1,2-a]pyrazin-7(8H)-yl)prop-2-en-1-one; or
1-(3-(2,3′-dichloro-2′,6-difluoro-6′-hydroxy-[1,1′-biphenyl]-4-yl)-2-(2-isopropylphenyl)-5,6-dihydroimidazo[1,2-a]pyrazin-7(8H)-yl)prop-2-en-1-one;
14)1-(2-(2-Isopropylphenyl)-3-(2,3′,5′-trichloro-2′,6-difluoro-6′-hydroxy-[1,1′-biphenyl]-4-yl)-5,6-dihydroimidazo[1,2-a]pyrazin-7(8H)-yl)prop-2-en-1-one;
15)1-(3-(2,3′-Dichloro-6,6′-difluoro-2′-hydroxy-[1,1′-biphenyl]-4-yl)-5,6-dihydroimidazo [1,2-a]pyrazin-7(8H)-yl)prop-2-en-1-one;
16) 1-(3-(2,3′-dichloro-6′-fluoro-2′-hydroxy-6-(2,2,2-trifluoroethoxy)-[1,1′-biphenyl]-4-yl)-5,6-dihydro-[1,2,4]triazolo[4,3-a]pyrazin-7(8H)-yl)prop-2-en-1-one;
17) 1-(3-(2-chloro-2′-fluoro-6′-hydroxy-6-(2,2,2-trifluoroethoxy)-[1,1′-biphenyl]-4-yl)-5,6-dihydro-[1,2,4]triazolo[4,3-a]pyrazin-7(8H)-yl)prop-2-en-1-one; and
18)1-(3-(2,3′-Dichloro-6,6′-difluoro-2′-hydroxy-[1,1′-biphenyl]-4-yl)-2-(trifluoromethyl)-5,6-dihydroimidazo[1,2-a]pyrazin-7(8H)-yl)prop-2-en-1-one.

The present invention also provides a pharmaceutical composition, comprising a therapeutically effective amount of at least one compound represented by formula (I) and at least one pharmaceutically acceptable carrier.

The present invention further provides a pharmaceutical composition, wherein the weight ratio of the compound represented by formula (I) to the pharmaceutically acceptable carrier is 0.0001:1-10.

The present invention provides the application of the compound or pharmaceutical composition represented by the structural formula (I) in the preparation of medicament.

The present invention further provides a preferred technical solution for the application:

In a further embodiment, the above-mentioned application is an application in the preparation of a medicament for treating and/or preventing cancer.

In a further embodiment, the use is an application for preparing a medicine for treating diseases mediated by KRAS G12C.

In a further embodiment, the disease is cancer.

In a further embodiment, the cancer is selected from the group consisting of breast cancer, multiple myeloma, bladder cancer, endometrial cancer, gastric cancer, cervical cancer, rhabdomyosarcoma, non-small cell lung cancer, small cell lung cancer, pleomorphic lung cancer, ovarian cancer, esophagus cancer, melanoma, colorectal cancer, hepatocellular carcinoma, head and neck tumor, hepatobiliary cell carcinoma, myelodysplastic syndrome, malignant glioma, prostate cancer, thyroid cancer, xuwang's cell tumor, lung squamous cell carcinoma, lichenoid keratosis, synovial sarcoma, skin cancer, pancreatic cancer, testicular cancer and liposarcoma.

The present invention also provides a method for treating and/or preventing diseases mediated by KRAS G12C, which comprises administering a therapeutically effective amount of at least any one compound represented by structural formula (I) or a pharmaceutical composition containing the same to a subject.

The present invention also provides a method for treating cancer, which comprises administering a therapeutically effective amount of at least any one of the compounds represented by structural formula (I) or a pharmaceutical composition containing the same to the subject.

In a further embodiment, in the above method, the KRAS G12C-mediated disease is cancer.

In a further embodiment, in the above-mentioned method, the cancer is selected from the group consisting of breast cancer, multiple myeloma, bladder cancer, endometrial cancer, gastric cancer, cervical cancer, rhabdomyosarcoma, non-small cell lung cancer, small cell lung cancer, pleomorphic lung cancer, ovarian cancer, esophageal cancer, melanoma, colorectal cancer, hepatocellular tumor, head and neck tumor, hepatobiliary cell carcinoma, myelodysplastic syndrome, malignant glioma, prostate cancer, thyroid cancer, xuwang's cell tumor, lung squamous cell carcinoma, lichenoid keratosis, synovial sarcoma, skin cancer, pancreatic cancer, testicular cancer and liposarcoma.

Unless otherwise specified, the general chemical terms used in the general structural formula have their usual meanings.

For example, unless otherwise specified, the term “halogen” used in the present invention refers to fluorine, chlorine, bromine or iodine.

In the present invention, unless otherwise specified, “alkyl” will be understood to mean a linear or branched monovalent saturated hydrocarbon group. For example, alkyl includes methyl, ethyl, propyl, isopropyl, n-butyl, isobutyl, sec-butyl, tert-butyl, n-pentyl, 3-(2-methyl)butyl, 2-pentyl, 2-methylbutyl, neopentyl, n-hexyl, 2-hexyl, 2-methylpentyl, etc. Similarly, the “₁₋₈” in “C₁₋₈ alkyl” refers to a straight-chain or branched group containing 1, 2, 3, 4, 5, 6, 7 or 8 carbon atoms.

“C₁₋₂ alkylene” refers to methylene or 1,2-ethylene.

“Alkoxy” refers to the oxyether form of the aforementioned linear or branched alkyl group, which is —O-alkyl.

In the present invention, “a”, “an”, “the”, “at least one” and “one or more” can be used interchangeably. Thus, for example, a composition comprising “a” pharmaceutically acceptable excipient can be interpreted to mean that the composition includes “one or more” pharmaceutically acceptable excipients.

The term “aryl” in the present invention, unless otherwise specified, will be understood to mean an unsubstituted or substituted monocyclic or condensed ring aromatic group including carbon ring atoms. In a further embodiment, the aryl group is a 6 to 10 membered monocyclic or bicyclic aromatic ring group. In a further embodiment, it is phenyl and naphthyl. Most preferred is phenyl.

The term “heterocyclyl”, as used herein, unless otherwise specified, will be understood to mean an unsubstituted or substituted 3-10 membered stable monocyclic ring consisting of carbon atoms and 1-3 heteroatoms selected from N, O or S. The system in which nitrogen or sulfur heteroatoms can be selectively oxidized, and nitrogen heteroatoms can be selectively quaternized. The heterocyclyl can be attached to any heteroatom or carbon atom to form a stable structure. Examples of these heterocyclyls include, but are not limited to azetidinyl, pyrrolidinyl, piperidinyl, piperazinyl, oxopiperazinyl, oxopiperidinyl, tetrahydrofuranyl, dioxolane, tetrahydroimidazolyl, tetrahydrothiazolyl, tetrahydrooxazolyl, tetrahydropyranyl, morpholinyl, thiomorpholinyl, thiomorpholinyl sulfoxide, thiomorpholinylsulfone and tetrahydro oxadiazolyl.

The term “heteroaryl”, as used herein, unless otherwise specified, will be understood to mean an unsubstituted or substituted stable 5- or 6-membered monocyclic aromatic ring system or an unsubstituted or substituted 9- or 10-membered benzo-fused heteroaromatic ring system or bicyclic heteroaromatic ring system, which consists of carbon atoms and 1-4 heteroatoms selected from N, O or S, and wherein the nitrogen or sulfur heteroatoms can be selectively oxidized. The nitrogen heteroatoms can be selectively quaternized. The heteroaryl group can be attached to any heteroatom or carbon atom to form a stable structure. Examples of heteroaryl groups include, but are not limited to thienyl, furyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzothiazolyl, benzothiazolyl, benzene and thiadiazolyl, benzotriazolyl adenine, quinolinyl or isoquinolinyl.

The term “cycloalkyl” will be understood to mean a cyclic saturated alkyl chain having 3-10 carbon atoms, for example, cyclopropyl, cyclobutyl, cyclopentyl or cyclohexyl.

The term “substituted” will be understood to mean that one or more hydrogen atoms in the group are replaced by the same or different substituents. Typical substituents include, but are not limited to halogen (F, Cl, Br or I), C₁₋₈ alkyl, C₃₋₁₂ cycloalkyl, —OR₁, —SR₁, ═O, ═S, —C(O)R₁, —C(S)R₁, ═NR₁, —C(O)OR₁, —C(S)OR₁, —NR₁R₂, —C(O)NR₁R₂, cyano, nitro, —S(O)₂R₁, —OS (O₂) OR₁, —OS(O)₂R₁, —OP(O)(OR₁)(OR₂). Wherein R₁ and R₂ are independently selected from —H, C₁₋₆ alkyl, and C₁₋₆ haloalkyl. In a further aspect, the substituents are independently selected from the group comprising —F, —Cl, —Br, —I, —OH, trifluoromethoxy, ethoxy, propoxy, isopropoxy, n-butoxy group, isobutoxy, tert-butoxy, —SCH₃, —SC₂H₅, formaldehyde, —C(OCH₃), cyano, nitro, —CF₃, —OCF₃, amino, dimethylamino, methylthio, sulfonyl and acetyl groups.

Examples of substituted alkyl groups include, but are not limited to, 2,3-dihydroxypropyl, 2-aminoethyl, 2-hydroxyethyl, pentachloroethyl, trifluoromethyl, methoxymethyl, pentafluoroethyl, phenylmethyl, dioxenylmethyl and piperazinylmethyl.

Examples of substituted alkoxy groups include, but are not limited to, 2-hydroxyethoxy, 2-fluoroethoxy, 2,2-difluoroethoxy, 2-methoxyethoxy, 2-aminoethoxy, 2,3-dihydroxypropoxy, cyclopropylmethoxy, aminomethoxy, trifluoromethoxy, 2-diethylaminoethoxy, 2-ethoxycarbonylethoxy, 3-hydroxypropoxy.

The term “pharmaceutically acceptable salt” will be understood to mean a salt prepared from a pharmaceutically acceptable non-toxic base or acid.

Since the compound represented by formula (I) will be used as a medicine, it is preferable to use a certain purity, for example, at least 60% purity, more suitable purity is at least 75%, and particularly suitable purity is at least 98% (% is weight ratio).

The prodrug of the compound of the present invention is included in the protection scope of the present invention. Generally, the prodrug refers to a functional derivative that is easily converted into a desired compound in the body. For example, any pharmaceutically acceptable salt, ester, salt of ester or other derivative of the compound of the present application can directly or indirectly provide the compound of the present application or its pharmaceutically active metabolite or residues.

The compound of the present invention may contain one or more asymmetric centers, and may produce diastereomers and optical isomers from this. The present invention includes all possible diastereomers and their racemic mixtures, their substantially pure resolved enantiomers, all possible geometric isomers and their pharmaceutically acceptable salts.

The above formula (I) does not exactly define the three-dimensional structure of a certain position of the compound. The present invention includes all stereoisomers of the compound represented by formula (I) and pharmaceutically acceptable salts thereof. Further, mixtures of stereoisomers and specific isolated stereoisomers are also included in the present invention. In the synthetic process of preparing such compounds, or in the process of racemization or epimerization known to those skilled in the art, the product obtained may be a mixture of stereoisomers.

When the compound represented by formula (I) and its pharmaceutically acceptable salt have solvates or polymorphs, the present invention includes any possible solvates and polymorphs. The type of solvent that forms the solvate is not particularly limited, as long as the solvent is pharmaceutically acceptable. For example, water, ethanol, propanol, acetone and similar solvents can be used.

The term “composition”, as used herein, will be understood to mean a product comprising a specified amount of each specified ingredient, and any product produced directly or indirectly from a combination of specified amounts of each specified ingredient. Therefore, pharmaceutical compositions containing the compounds of the present invention as active ingredients and methods for preparing the compounds of the present invention are also part of the present invention. In addition, some crystalline forms of the compound may exist in polymorphs, and this polymorph is included in the present invention. In addition, some compounds can form solvates with water (ie, hydrates) or common organic solvents, and such solvates also fall within the scope of the present invention.

The pharmaceutical composition provided by the present invention includes as an active component a compound represented by formula (I) (or a pharmaceutically acceptable salt thereof), a pharmaceutically acceptable excipient and other optional therapeutic components or accessories. Although in any given case, the most suitable way of administering the active ingredient depends on the particular subject to be administered, the nature of the subject and the severity of the disease, the pharmaceutical composition of the present invention includes oral, rectal, topical and a pharmaceutical composition for parenteral administration (including subcutaneous administration, intramuscular injection, and intravenous administration). The pharmaceutical composition of the present invention can be conveniently prepared in a unit dosage form known in the art and prepared by any preparation method known in the pharmaceutical field.

In general, to treat the conditions or discomforts shown above, the dose level of the drug is about 0.01 mg/kg body weight to 150 mg/kg body weight per day, or 0.5 mg to 7 g per patient per day. For example, inflammation, cancer, psoriasis, allergies/asthma, diseases and discomforts of the immune system, diseases and discomforts of the central nervous system (CNS), the effective treatment drug dosage level is 0.01 mg/kg body weight to 50 mg/kg body weight per day, or 0.5 mg to 3.5 g per patient per day.

However, it is understood that lower or higher dosages than those mentioned above may be required. The specific dosage level and treatment plan for any particular patient will depend on many factors, including the activity of the specific compound used, age, weight, overall health, gender, diet, administration time, administration route, excretion rate, and the condition of drug combination and the severity of the specific disease being treated.

Typical compounds of the present invention include, but are not limited to the compounds shown in Table 1:

TABLE 1

| Example | Chemical structure and name |
|---|---|
| 1 | 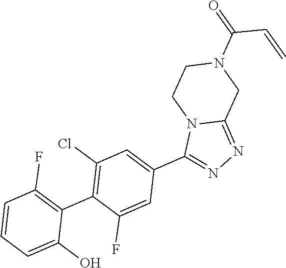 [SMILES: C=CC(=O)N1CCn2c(nnc2-c2cc(F)c(-c3c(O)cccc3F)c(Cl)c2)C1] |
| | 1-(3-(2-chloro-2′,6-difluoro-6′-hydroxy-[1,1′-biphenyl]-4-yl)-5,6-dihydro-[1,2,4]triazolo[4,3-a]pyrazin-7(8 |
| | H)-yl)prop-2-en-1-one; |
| 2 | 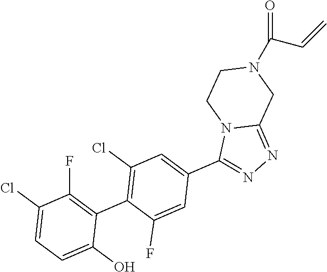 [SMILES: C=CC(=O)N1CCn2c(nnc2-c2cc(F)c(-c3c(O)ccc(Cl)c3F)c(Cl)c2)C1] |
| | 1-(3-(2,3′-dichloro-2′,6-difluoro-6′-hydroxy-[1,1′-biphenyl]-4-yl)-5,6-dihydro-[1,2,4]triazolo[4,3-a]pyrazin- |
| | 7(8H)-yl)prop-2-en-1-one; |
| 2′ | 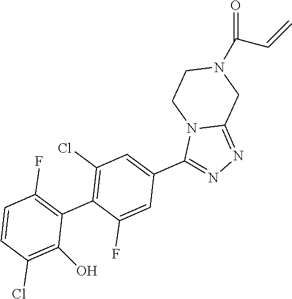 [SMILES: C=CC(=O)N1CCn2c(nnc2-c2cc(F)c(-c3c(F)ccc(Cl)c3O)c(Cl)c2)C1] |
| | 1-(3-(2,3′-dichloro-6,6′-difluoro-2′-hydroxy-[1,1′-biphenyl]-4-yl)-5,6-dihydro-[1,2,4]triazolo[4,3-a]pyrazin- |
| | 7(8H)-yl)prop-2-en-1-one; |
| 3 | 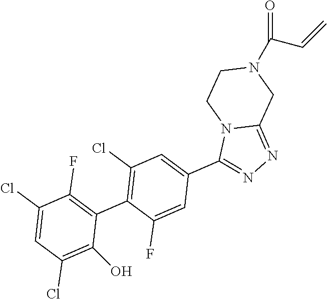 [SMILES: C=CC(=O)N1CCn2c(nnc2-c2cc(F)c(-c3c(O)c(Cl)cc(Cl)c3F)c(Cl)c2)C1] |
| | 1-(3-(2,3′,5′-trichloro-2′,6-difluoro-6′-hydroxy-[1,1′-biphenyl]-4-yl)-5,6-dihydro-[1,2,4]triazolo[4,3-a]pyrazin- |
| | 7(8H)-yl)prop-2-en-1-one; |
| 4 | 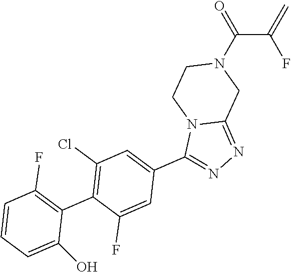 [SMILES: C=C(F)C(=O)N1CCn2c(nnc2-c2cc(F)c(-c3c(O)cccc3F)c(Cl)c2)C1] |
| | 1-(3-(2-chloro-2′,6-difluoro-6′-hydroxy-[1,1′-biphenyl]-4-yl)-5,6-dihydro-[1,2,4]triazolo[4,3-a]pyrazin-7(8 |
| | H)-yl)-2-fluoroprop-2-en-1-one; |
| 5 | 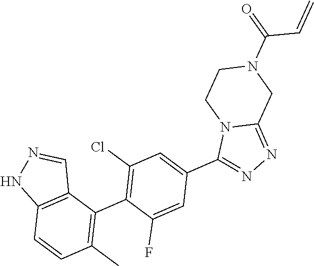 [SMILES: C=CC(=O)N1CCn2c(nnc2-c2cc(F)c(-c3c(C)ccc4[nH]ncc34)c(Cl)c2)C1] |
| | 1-(3-(3-chloro-5-fluoro-4-(5-methyl-1H-indazol-4-yl)phenyl)-5,6-dihydro-[1,2,4]triazolo[4,3-a]pyrazin-7(8 |
| | H)-yl)prop-2-en-1-one; |
| 6 | 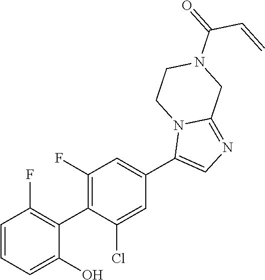 [SMILES: C=CC(=O)N1CCn2c(-c3cc(F)c(-c4c(O)cccc4F)c(Cl)c3)cnc2C1] |
| | 1-(3-(2-chloro-2′,6-difluoro-6′-hydroxy-[1,1′-biphenyl]-4-yl)-5,6-dihydroimidazo[1,2-a]pyrazin-7(8H)-yl) |
| | prop-2-en-1-one; |
| 7 | 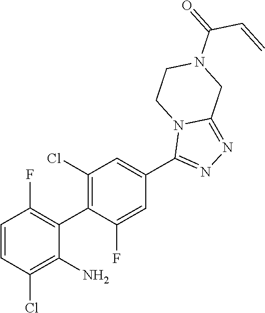 [SMILES: C=CC(=O)N1CCn2c(nnc2-c2cc(F)c(-c3c(F)ccc(Cl)c3N)c(Cl)c2)C1] |
| | 1-(3-(2′-amino-2,3′-dichloro-6,6′-difluoro-[1,1′-biphenyl]-4-yl)-5,6-dihydro-[1,2,4]triazolo[4,3-a]pyrazin-7 |
| | (8H)-yl)prop-2-en-1-one; |
| 8 | 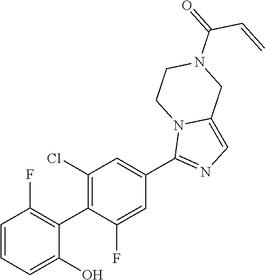 [SMILES: C=CC(=O)N1CCn2c(cnc2-c2cc(F)c(-c3c(O)cccc3F)c(Cl)c2)C1] |
| | 1-(3-(2-chloro-2′,6-difluoro-6′-hydroxy-[1,1′-biphenyl]-4-yl)-5,6-dihydroimidazo[1,5-a]pyrazin-7(8H)-yl)prop- |
| | 2-en-1-one; |
| 9 | 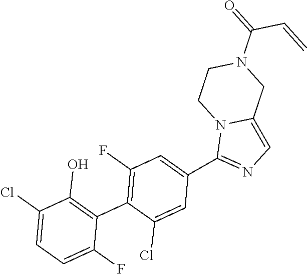 [SMILES: C=CC(=O)N1CCn2c(cnc2-c2cc(F)c(-c3c(F)ccc(Cl)c3O)c(Cl)c2)C1] |
| | 1-(3-(2,3′-dichloro-6,6′-difluoro-2′-hydroxy-[1,1′-biphenyl]-4-yl)-5,6-dihydroimidazo[1,5-a]pyrazin-7(8H)- |
| | yl)prop-2-en-1-one; |
| 9′ | 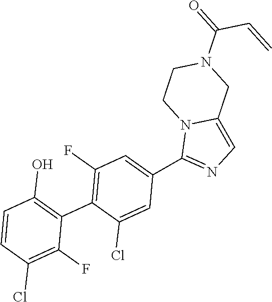 [SMILES: C=CC(=O)N1CCn2c(cnc2-c2cc(F)c(-c3c(O)ccc(Cl)c3F)c(Cl)c2)C1] |
| | 1-(3-(2,3′-dichloro-2′,6-difluoro-6′-hydroxy-[1,1′-biphenyl]-4-yl)-5,6-dihydroimidazo[1,5-a]pyrazin-7(8H)- |
| | yl)prop-2-en-1-one; |
| 10 | 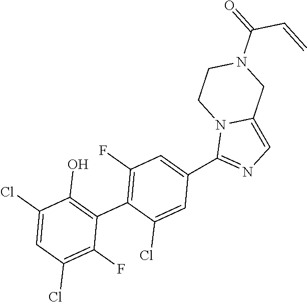 [SMILES: C=CC(=O)N1CCn2c(cnc2-c2cc(F)c(-c3c(O)c(Cl)cc(Cl)c3F)c(Cl)c2)C1] |
| | 1-(3-(2,3′,5′-trichloro-2′,6-difluoro-6′-hydroxy-[1,1′-biphenyl]-4-yl)-5,6-dihydroimidazo[1,5-a]pyrazin-7(8 |
| | H)-yl)prop-2-en-1-one; |
| 11 | 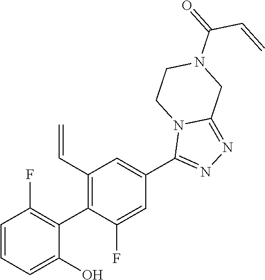 [SMILES: C=CC(=O)N1CCn2c(nnc2-c2cc(F)c(-c3c(O)cccc3F)c(C=C)c2)C1] |
| | 1-(3-(2,2′-difluoro-6′-hydroxy-6-vinyl-[1,1′-biphenyl]-4-yl)-5,6-dihydro-[1,2,4]triazolo[4,3-a]pyrazin-7(8H)- |
| | yl)prop-2-en-1-one; |
| 12 | 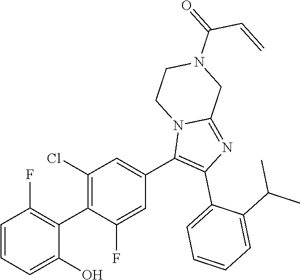 [SMILES: C=CC(=O)N1CCn2c(nc(-c3ccccc3C(C)C)c2-c2cc(F)c(-c3c(O)cccc3F)c(Cl)c2)C1] |
| | 1-(3-(2-chloro-2′,6-difluoro-6′-hydroxy-[1,1′-biphenyl]-4-yl)-2-(2-isopropylphenyl)-5,6-dihydroimidazo[1, |
| | 2-a]pyrazin-7(8H)-yl)prop-2-en-1-one; |
| 13 | 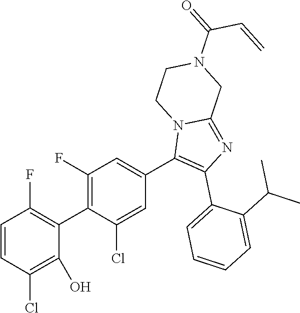 [SMILES: C=CC(=O)N1CCn2c(nc(-c3ccccc3C(C)C)c2-c2cc(F)c(-c3c(F)ccc(Cl)c3O)c(Cl)c2)C1] |
| | 1-(3-(2,3′-dichloro-6,6′-difluoro-2′-hydroxy-[1,1′-biphenyl]-4-yl)-2-(2-isopropylphenyl)-5,6-dihydroimidazo |
| | [1,2-a]pyrazin-7(8H)-yl)prop-2-en-1-one; |
| 13′ | 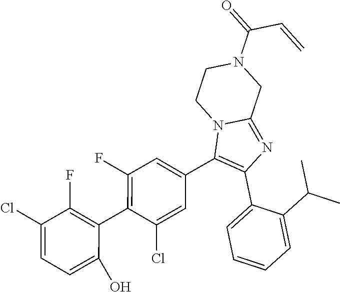 [SMILES: C=CC(=O)N1CCn2c(nc(-c3ccccc3C(C)C)c2-c2cc(F)c(-c3c(O)ccc(Cl)c3F)c(Cl)c2)C1] |
| | 1-(3-(2,3′-dichloro-2′,6-difluoro-6′-hydroxy-[1,1′-biphenyl]-4-yl)-2-(2-isopropylphenyl)-5,6-dihydroimidazo |
| | [1,2-a]pyrazin-7(8H)-yl)prop-2-en-1-one; |
| 14 | 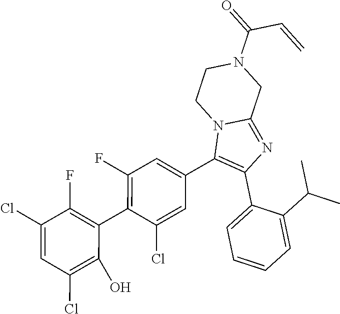 [SMILES: C=CC(=O)N1CCn2c(nc(-c3ccccc3C(C)C)c2-c2cc(F)c(-c3c(O)c(Cl)cc(Cl)c3F)c(Cl)c2)C1] |
| | 1-(2-(2-isopropylphenyl)-3-(2,3′,5′-trichloro-2′,6-difluoro-6′-hydroxy-[1,1′-biphenyl]-4-yl)-5,6-dihydroimidazo |
| | [1,2-a]pyrazin-7(8H)-yl)prop-2-en-1-one; |
| 15 | 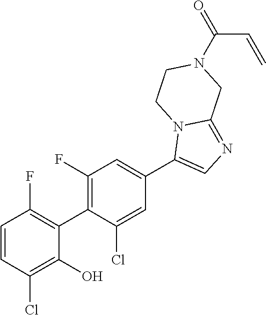 [SMILES: C=CC(=O)N1CCn2c(-c3cc(F)c(-c4c(F)ccc(Cl)c4O)c(Cl)c3)cnc2C1] |
| | 1-(3-(2,3′-dichloro-6,6′-difluoro-2′-hydroxy-[1,1′-biphenyl]-4-yl)-5,6-dihydroimidazo[1,2-a]pyrazin-7(8H)- |
| | yl)prop-2-en-1-one; |
| 16 | 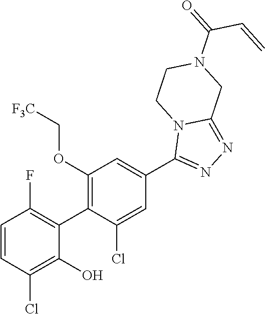 [SMILES: C=CC(=O)N1CCn2c(nnc2-c2cc(Cl)c(-c3c(F)ccc(Cl)c3O)c(OCC(F)(F)F)c2)C1] |
| | 1-(3-(2,3′-dichloro-6′-fluoro-2′-hydroxy-6-(2,2,2-trifluoroethoxy)-[1,1′-biphenyl]-4-yl)-5,6-dihydro-[1,2,4] |
| | triazolo[4,3-a]pyrazin-7(8H)-yl)prop-2-en-1-one; |
| 17 | 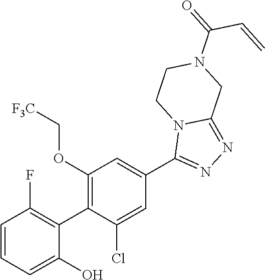 [SMILES: C=CC(=O)N1CCn2c(nnc2-c2cc(Cl)c(-c3c(O)cccc3F)c(OCC(F)(F)F)c2)C1] |
| | 1-(3-(2-chloro-2′-fluoro-6′-hydroxy-6-(2,2,2-trifluoroethoxy)-[1,1′-biphenyl]-4-yl)-5,6-dihydro-[1,2,4]triazolo |
| | [4,3-a]pyrazin-7(8H)-yl)prop-2-en-1-one; |
| 18 | 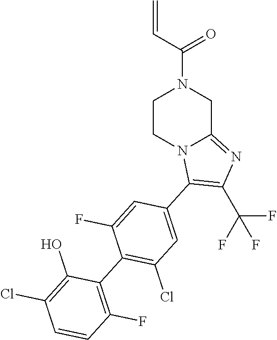 [SMILES: C=CC(=O)N1CCn2c(nc(C(F)(F)F)c2-c2cc(F)c(-c3c(F)ccc(Cl)c3O)c(Cl)c2)C1] |
| | 1-(3-(2,3′-dichloro-6,6′-difluoro-2′-hydroxy-[1,1′-biphenyl]-4-yl)-2-(trifluoromethyl)-5,6-dihydroimidazo |
| | [1,2-a]pyrazin-7(8H)-yl)prop-2-en-1-one; |

### EXAMPLES

In order to make the above content clearer and clearer, the present invention will use the following embodiments to further illustrate the technical solution of the present invention. The following examples are only used to illustrate specific implementations of the present invention, so that those skilled in the art can understand the present invention, but are not used to limit the protection scope of the present invention. In the specific embodiments of the present invention, technical means or methods that are not specifically described are conventional technical means or methods in the art.

Unless otherwise specified, all parts and percentages in the present invention are calculated by weight, and all temperatures refer to ° C.

The following abbreviations have been used:

DCM: dichloromethane;

Dioxane: Dioxane

EA: ethyl acetate;

PE: petroleum ether;

MeOH: methanol;

TFA: trifluoroacetic acid;

DIEA: N,N-diisopropylethylamine;

Pd(dppf)₂Cl₂: [1,1′-bis(diphenylphosphino)ferrocene]palladium dichloride;

Sphos Pd G2: Chloro(2-dicyclohexylphosphino-2′,6′-dimethoxy-1,1′-biphenyl)(2′-amino-1,1′-biphenyl-2-base) palladium(II);

CDCl₃: deuterated chloroform;

pre-TLC: Thin layer chromatography silica gel plate;

pre-HPLC: High performance liquid chromatography.

### Example 1: Compound 1-(3-(2-Chloro-2′,6-difluoro-6′-hydroxy-[1,1′-biphenyl]-4-yl)-5,6-dihydro-[1,2,4]triazolo[4,3-a]pyrazin-7(8H)-yl)prop-2-en-1-one

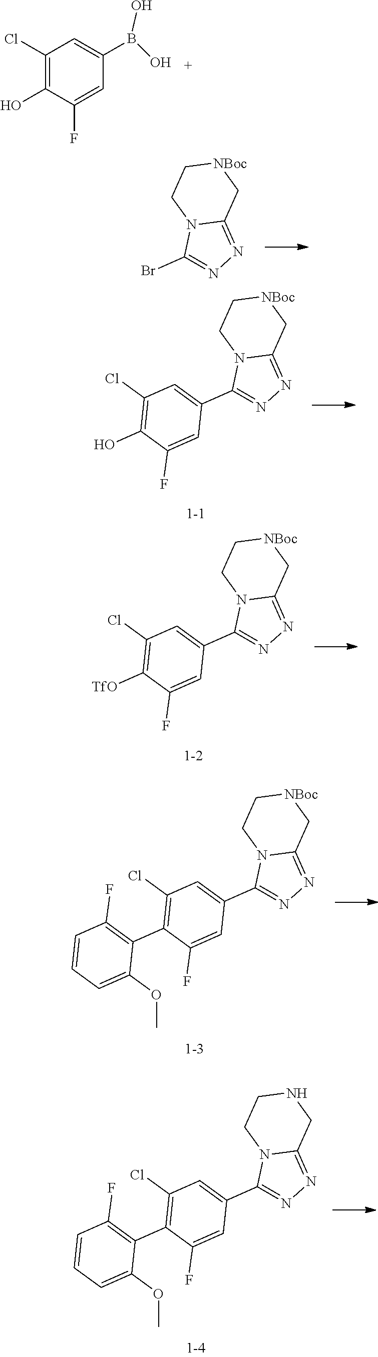

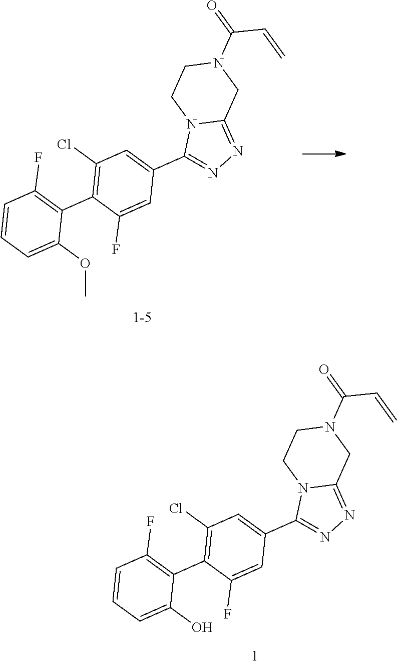 [SMILES: C=CC(=O)N1CCn2c(nnc2-c2cc(F)c(-c3c(F)cccc3OC)c(Cl)c2)C1 ; C=CC(=O)N1CCn2c(nnc2-c2cc(F)c(-c3c(O)cccc3F)c(Cl)c2)C1]

### Step 1: Synthesis of Compound 1-1

In a 20 ml microwave tube, (3-chloro-5-fluoro-4-hydroxyphenyl)boronic acid (500 mg), 3-bromo-6,8-dihydro-5H-[1,2,4] triazolo[4,3-a]pyrazine-7-carboxylate tert-butyl ester (875.87 mg), Pd(dppf)Cl₂ (192.00 mg), and cesium carbonate (1.71 g) are added to the mixed solution of dioxane (1.5 mL) and H₂O (0.1 mL) at room temperature, after the reaction liquid was replaced with nitrogen, and then reacted at 90° C. for 1 h. The reaction was poured into DCM, dried, filtered and concentrated. The concentrate was purified by silica gel column chromatography (MeOH:DCM=0-4%) to give the desired product (450 mg, 46.46% yield) as a white solid, namely compound 1-1. ESI-MS m/z: 369.26 [M+H]⁺.

### Step 2: Synthesis of Compounds 1-2

Compound 1-1 (419 mg) and DIEA (229.93 mg) were dissolved in DCM (3 mL) at room temperature, then 1,1,1-trifluoro-N-phenyl-N-(trifluoromethane) sulfonyl)methanesulfonic acid (608.83 mg) was added with stirring, and continued stirring at room temperature for 1 h. The compound was directly concentrated, dissolved in a small amount of DCM, and purified by pre-TLC (EA) to give the desired product (380 mg, 66.78% yield) as a white solid, namely compound 1-2. ESI-MS m/z: 501.09 [M+H]⁺.

### Step 3: Synthesis of Compounds 1-3

At room temperature, compound 1-2 (160 mg), (2-fluoro-6-methoxyphenyl)boronic acid (54.29 mg), S-phos Pd G2 (31.95 umol), and K₃PO₄ (638.91 umol) were added to the solution of dioxane (2 mL) in a 10 ml microwave tube, the reaction solution was replaced with nitrogen, and then the reaction was microwaved at 80° C. for 40 min. The reaction was poured into DCM, dried, filtered and concentrated. The concentrate was purified by pre-TLC to obtain the target product (100 mg, 65.64% yield) as a white solid, namely compound 1-3. ESI-MS m/z: 477.25 [M+H]⁺.

### Step 4: Synthesis of Compounds 1-4

Compound 1-3 (95 mg) was dissolved in a mixture of TFA (2 mL) and DCM (6 mL) at room temperature, and then the reaction mixture was stirred at room temperature for 10 min. An appropriate amount of toluene was added, followed by concentration to obtain the target product (70 mg) as a pale yellow oil, namely compound 1-4. ESI-MS m/z: 377.25 [M+H]⁺.

### Step 5: Synthesis of Compounds 1-5

Compound 1-4 (69 mg) and DIEA (71.00 mg) were dissolved in DCM (5 mL) at room temperature, then the reaction mixture cooled to 0° C., acryloyl chloride (21.55 mg) was added with stirring, and stirring was continued for 5 min. To the mixture was added DCM and ice water, the organic phase was dried over anhydrous sodium sulfate, filtered and concentrated. The concentrate was purified by pre-TLC (DCM:MeOH=10:1) to give the target product (51 mg, 64.64% yield) as a white solid namely compound 1-5. ESI-MS m/z: 431.26 [M+H]⁺.

### Step 6: Synthesis of Compound 1

At room temperature, compound 1-5 (50 mg) was dissolved in DCM (10 mL), then the reaction mixture was cooled to 0° C., BBr₃ (435.20 mg) was added with stirring, and then the reaction mixture was slowly returned to room temperature and stirred for 30 min. At 0° C., saturated sodium bicarbonate solution was added dropwise with stirring to quench BBr₃, then DCM was added, and the organic phase was dried over anhydrous sodium sulfate, filtered, and concentrated. The concentrate was purified by pre-HPLC to give the target product (0.8 mg, 1.65% yield, 95.07% purity) as a white solid, namely compound 1.ESI-MS m/z: 431.26 [M+H]⁺. ¹H NMR (500 MHz, DMSO-d₆)δ7.84 (s, 1H), 7.72 (d, J=9.4 Hz, 1H), 7.34 (q, J=7.9 Hz, 1H), 7.07-6.92 (m, 1H), 6.89-6.68 (m, 2H), 6.24 (dd, J=16.5, 2.2 Hz, 1H), 5.82 (d, J=10.4 Hz, 1H), 5.14 (s, 1H), 4.96 (s, 1H), 4.31 (d, J=18.2 Hz, 2H), 4.01 (d, J=30.3 Hz, 2H).

### Example 2: Compound 1-(3-(2,3′-dichloro-6,6′-difluoro-2′-hydroxy-[1,1′-biphenyl]-4-yl)-5,6-dihydro-[1,2,4]triazolo[4,3-a]pyrazin-7(8H)-yl)prop-2-en-1-one and/or 1-(3-(2,3′-dichloro-2′,6-difluoro-6′-hydroxy-[1,1′-biphenyl]-4-yl)-5,6-dihydro-[1,2,4]trisazolo[4,3-a]pyrazin-7(8H)-yl)prop-2-en-1-one

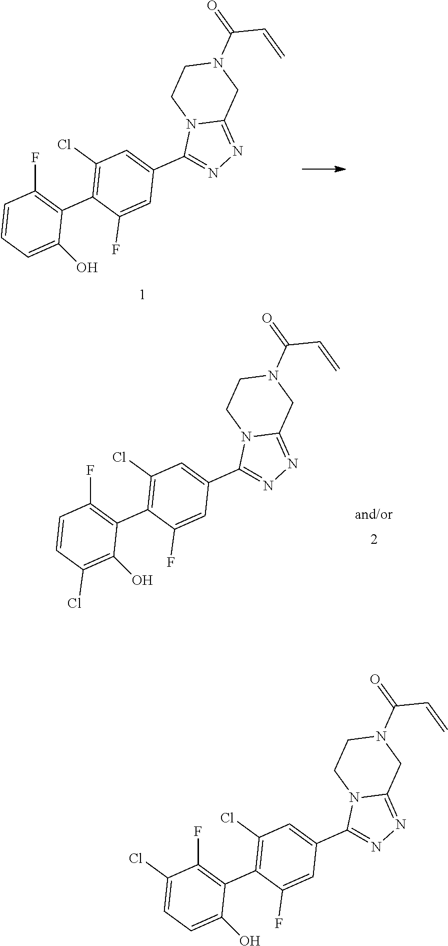 [SMILES: C=CC(=O)N1CCn2c(nnc2-c2cc(F)c(-c3c(O)cccc3F)c(Cl)c2)C1 ; C=CC(=O)N1CCn2c(nnc2-c2cc(F)c(-c3c(F)ccc(Cl)c3O)c(Cl)c2)C1 ; C=CC(=O)N1CCn2c(nnc2-c2cc(F)c(-c3c(O)ccc(Cl)c3F)c(Cl)c2)C1]

Compound 1 (20 mg) was dissolved in acetic acid (2 mL) at room temperature, 1-chloropyrrolidine-2,5-dione (6.41 mg) was added with stirring, and stirring was continued for 12 hours. Ethyl acetate was added to the reaction solution, followed by washing with water, and the organic phase was concentrated. The concentrate was purified by pre-HPLC to give the target product (0.8 mg, 3.69% yield, 96.10% purity) as a white solid, namely compound 2. ESI-MS m/z: 451.2 [M+H]⁺.

### Example 3: Compound 1-(3-(2,3′,5′-trichloro-2′,6-difluoro-6′-hydroxy-[1,1′-biphenyl]-4-yl)-5,6-dihydro-[1,2,4]triazolo[4,3-a]pyrazin-7(8H)-yl)prop-2-en-1-one

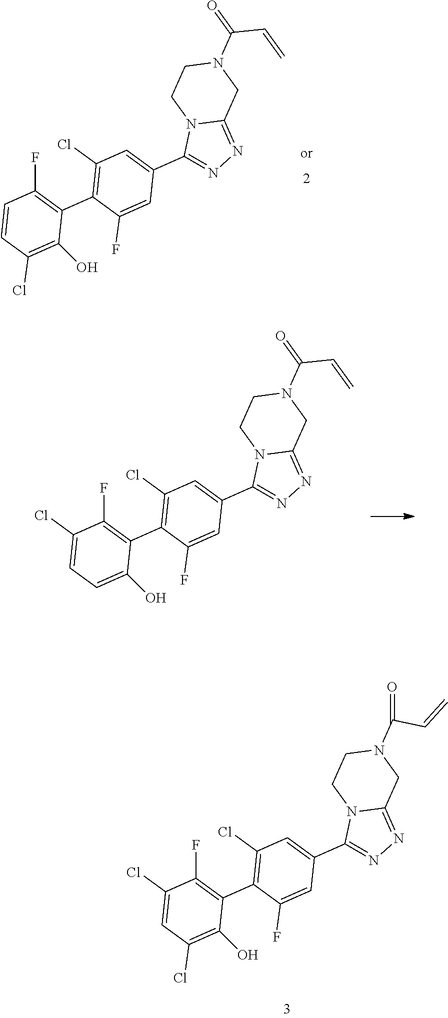 [SMILES: C=CC(=O)N1CCn2c(nnc2-c2cc(F)c(-c3c(F)ccc(Cl)c3O)c(Cl)c2)C1 ; C=CC(=O)N1CCn2c(nnc2-c2cc(F)c(-c3c(O)ccc(Cl)c3F)c(Cl)c2)C1 ; C=CC(=O)N1CCn2c(nnc2-c2cc(F)c(-c3c(O)c(Cl)cc(Cl)c3F)c(Cl)c2)C1]

Compound 2 (19 mg) was dissolved in acetic acid (1.5 mL) at room temperature, 1-chloropyrrolidine-2,5-dione (12.17 mg) was added with stirring, and the mixture was stirred at room temperature for 12 h. Ethyl acetate was added to the reaction solution, followed by washing with water, and the organic phase was concentrated. The concentrate was purified by pre-HPLC to give the target product (2.8 mg, 12.65% yield, 99.05% purity) as a white solid, namely compound 3. ESI-MS m/z: 485.1 [M+H]⁺.

### Example 4: Compound 1-(3-(2-Chloro-2′,6-difluoro-6′-hydroxy-[1,1′-biphenyl]-4-yl)-5,6-dihydro-[1,2,4]triazolo[4,3-a]pyrazin-7(8H)-yl)-2-fluoroprop-2-en-1-one

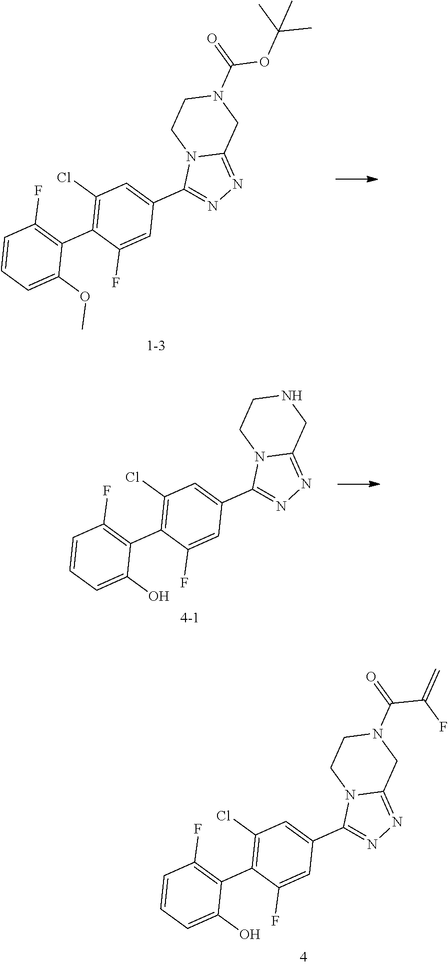 [SMILES: COc1cccc(F)c1-c1c(F)cc(-c2nnc3n2CCN(C(=O)OC(C)(C)C)C3)cc1Cl ; Oc1cccc(F)c1-c1c(F)cc(-c2nnc3n2CCNC3)cc1Cl ; C=C(F)C(=O)N1CCn2c(nnc2-c2cc(F)c(-c3c(O)cccc3F)c(Cl)c2)C1]

### Step 1: Synthesis of Compound 4-1

At room temperature, compound 1-3 (200 mg) was dissolved in DCM (8 mL), then the reaction mixture was cooled to 0° C., BBr₃ (420.25 mg) was added with stirring, and then the reaction mixture was slowly returned to room temperature and stirred for 12 h. At 0° C., saturated sodium bicarbonate solution was added dropwise with stirring to quench BBr₃, then DCM was added, and the organic phase was dried over anhydrous sodium sulfate, filtered, and concentrated. The concentrate was purified by pre-TLC (MeOH:DCM=1:11) to give the target product (90 mg, 59.16% yield) as a white solid compound 4-1. ESI-MS m/z: 363.23 [M+H]⁺.

### Step 2: Synthesis of Compound 4

Compound 4-1 (30 mg), 2-fluoropropene-2-enoic acid (7.45 mg) and DIEA (21.38 mg) were dissolved in DMF (3 mL) at room temperature, then the reaction mixture was cooled to 0° C., and HATU was added with stirring (18.72 mg), then the reaction mixture was slowly returned to room temperature and stirred for 30 min. Ethyl acetate was added to the reaction solution, the organic phase was washed with acid, alkali and water, the organic phases were combined, dried over anhydrous sodium sulfate, filtered and concentrated. The concentrate was purified by pre-TLC (MeOH:DCM=1:11) to give the desired product (1.3 mg, 3.62% yield, 96.08% purity) as a white solid, namely compound 4. ESI-MS m/z: 435.2 [M+H]⁺.

### Example 5: Compound 1-(3-(3-chloro-5-fluoro-4-(5-methyl-1H-indazol-4-yl)phenyl)-5,6-dihydro-[1,2,4]triazolo[4,3-a]pyrazin-7(8H)-yl)prop-2-en-1-one

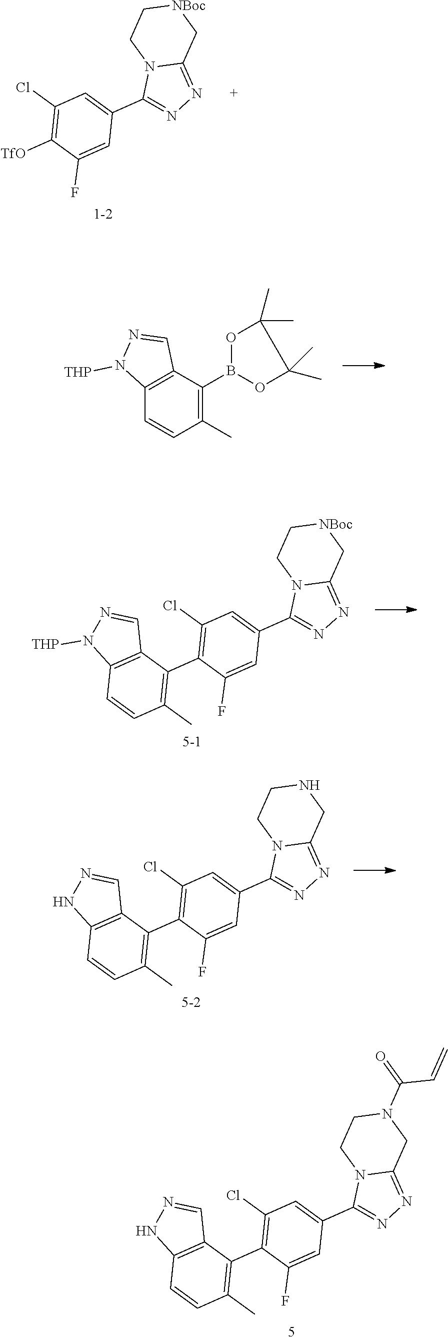

### Step 1: Synthesis of Compound 5-1

At room temperature, compound 1-2 (260 mg), 5-methyl-1-tetrahydropyran-2-yl-4-(4,4,5,5-tetramethyl-1,3,2-dioxin-2-yl)indazole (177.66 mg), S-phos Pd G2 (37.36 mg), and K₃PO₄ (220.39 mg) were added to a 10 ml microwave tube in dioxane (2.5 mL) solution, after the reaction solution was replaced with nitrogen, then microwave reaction at 85° C. for 50 min. The reaction solution was added to ethyl acetate, the organic phase was washed with water, dried over anhydrous sodium sulfate, filtered and concentrated. The concentrate was purified by pre-TLC (MeOH:DCM=1:12) to give the target product (52 mg, 17.67% yield) as a brown oil, namely compound 5-1. ESI-MS m/z: 567.32 [M+H]⁺.

### Step 2: Synthesis of Compound 5-2

Compound 5-1 (50 mg) was dissolved in a mixed solvent of DCM (2 mL) and TFA (1 mL) at room temperature, stirred at room temperature for 10 min, and then the reaction solution was heated to 40° C. and stirred for 1 h. The reaction solution was directly concentrated to obtain the target product (30 mg) as a brown oil, namely compound 5-2. ESI-MS m/z: 383.33 [M+H]⁺.

### Step 3: Synthesis of Compound 5

Compound 5-2 (30 mg) and DIEA (30.38 mg, 235.10 umol, 40.95 uL) were dissolved in DCM (2 mL) at room temperature, then cooled to 0° C., and acryloyl chloride (7.09 mg) was added with stirring. After the reaction was complete, the reaction solution was directly concentrated, and the concentrate was purified by pre-TLC and pre-HPLC to obtain the target product (7.8 mg, 22.78% yield, 87.51% purity) as a pale yellow solid, namely compound 5. ESI-MS m/z: 437.20 [M+H]⁺.

### Example 6: Compound 1-(3-(2-Chloro-2′,6-difluoro-6′-hydroxy-[1,1′-biphenyl]-4-yl)-5,6-dihydroimidazo [1,2-a]pyrazin-7(8H)-yl)prop-2-en-1-one

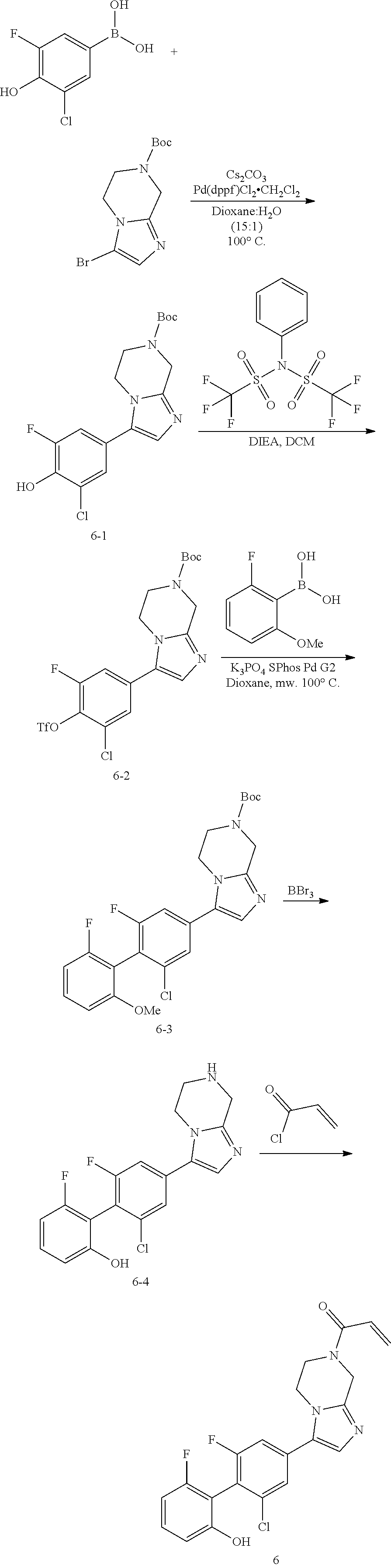

### Step 1: Synthesis of Compound 6-1

At room temperature, the compound 3-bromo-6,8-dihydro-5H-imidazo[1,2-a]pyrazine-7-carboxylate tert-butyl ester (400 mg), (3-chloro-5-fluoro-4-hydroxyphenyl) boronic acid (302.40 mg) and Cs₂CO₃ (646.96 mg) were dissolved in a mixed solvent of dioxane (15 mL) and H₂O (1 mL), under nitrogen protection, Pd(dppf)Cl₂.CH₂Cl₂ (54.05 mg) was added, and the reaction temperature was raised to 100° C. for 1.5 h. The reaction mixture was suctioned and filtered to remove insoluble matters, and the filtrate was concentrated. The concentrate was purified by pre-TLC (EA:MeOH=20:1) to obtain the target product (230 mg, 47.24% yield) as a pale yellow solid, namely compound 6-1.

### Step 2: Synthesis of Compound 6-2

Compound 6-1 (390 mg) and DIEA (548.17 mg) were dissolved in DCM (20 mL) at room temperature, and 1,1,1-trifluoro-N-phenyl-N-(trifluoromethane) sulfonyl)methanesulfonic acid (757.62 mg) was added after appropriate stirring, reacted at room temperature overnight. The reaction solution was directly concentrated to obtain the target product (500 mg, 61.32% yield) as a brown solid, namely compound 6-2.

### Step 3: Synthesis of Compound 6-3

At room temperature, under nitrogen atmosphere, compound 6-2 (50 mg), (2-fluoro-6-methoxyphenyl)boronic acid (20.40 mg), K₃PO₄ (42.46 mg) and S-phos Pd G2 (7.20 mg) were dissolved in dioxane (8 mL), and the reaction was microwaved at 70° C. for 40 min. The reaction mixture was suction filtered to remove insoluble matters, and the filtrate was concentrated. The concentrate was purified by pre-TLC (PE/EA=5:1) to obtain the target product (30 mg, 63.02% yield) as a white solid, namely compound 6-3. ESI-MS m/z: 476.24 [M+H]⁺.

### Step 4: Synthesis of Compounds 6-4

At room temperature, compound 6-3 (30 mg) was dissolved in DCM (9 mL), the mixture was cooled to 0° C. in an ice-water bath, BBr₃ (47.38 mg) was slowly added dropwise, and then the mixture was moved to room temperature to react for 1 h. At 0° C., saturated sodium bicarbonate solution was added dropwise with stirring to quench BBr₃, then DCM was added, and the organic phase was dried over anhydrous sodium sulfate, filtered and concentrated. The concentrate was purified by pre-TLC (EA:MeOH=4:1) to give the target product (20 mg, 87.70% yield) as a white solid, namely compound 6-4. ESI-MS m/z: 362.14 [M+H]⁺.

### Step 5: Synthesis of Compound 6

Compound 6-4 (20 mg) and DIEA (10.72 mg) were dissolved in DCM (5 mL) at room temperature, the mixture was cooled in an ice-water bath, acryloyl chloride (5.00 mg) was slowly added, and the reaction was stirred for 10 min. The reaction mixture was directly concentrated, and the concentrate was purified by pre-HPLC to give the target product (2.9 mg, 12.62% yield, 97% purity) as a white solid, namely compound 6. ESI-MS m/z: 416.29 [M+H]⁺.

### Example 7: Compound 1-(3-(2′-amino-2,3′-dichloro-6,6′-difluoro-[1,1′-biphenyl]-4-yl)-5,6-dihydro-[1,2,4]triazolo[4,3-a]pyrazin-7(8H)-yl)prop-2-en-1-one

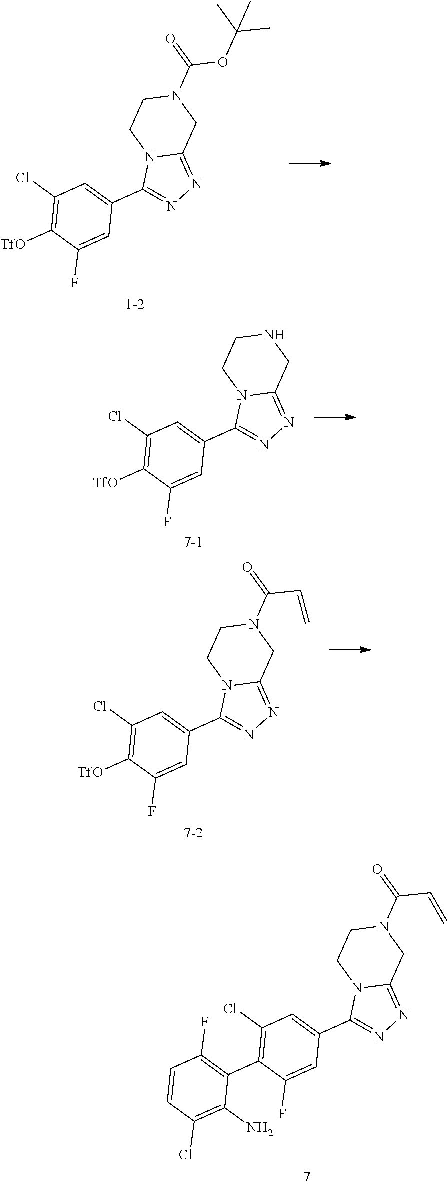 [SMILES: CC(C)(C)OC(=O)N1CCn2c(nnc2-c2cc(F)c(OS(=O)(=O)C(F)(F)F)c(Cl)c2)C1 ; O=S(=O)(Oc1c(F)cc(-c2nnc3n2CCNC3)cc1Cl)C(F)(F)F ; C=CC(=O)N1CCn2c(nnc2-c2cc(F)c(OS(=O)(=O)C(F)(F)F)c(Cl)c2)C1 ; C=CC(=O)N1CCn2c(nnc2-c2cc(F)c(-c3c(F)ccc(Cl)c3N)c(Cl)c2)C1]

### Step 1: Synthesis of Compound 7-1

Compound 1-2 (300 mg) was dissolved in DCM (5 mL) at room temperature, TFA (1 mL) was slowly added dropwise with stirring, and stirring was continued for 30 min. Five times the amount (V) of DCM was added to the reaction solution, followed by an appropriate amount of water, the reaction mixture was adjusted to weakly alkaline with saturated sodium bicarbonate solution, then the organic phase was washed with water, dried over anhydrous sodium sulfate, filtered and concentrated. The concentrate was purified by pre-TLC (DCM/MeOH=11:1) to give the desired product (210 mg, 87.49% yield) as a brown solid, namely compound 7-1. ESI-MS m/z: 401.16 [M+H]⁺.

### Step 2: Synthesis of Compound 7-2

Compound 7-1 (180 mg) was dissolved in DCM (5 mL) at room temperature, DIEA (116.10 mg) was added, the reaction mixture was moved to 0° C., acryloyl chloride (40.65 mg) was added with stirring, and stirring was continued for 3 min. An appropriate amount of DCM was added to the reaction solution, an appropriate amount of water was added, then sodium bicarbonate solution was added dropwise to adjust the pH to basicity, the organic phase was dried over anhydrous sodium sulfate, filtered and concentrated. The concentrate was purified by pre-TLC (DCM/MeOH=11:1) to give the desired product (141 mg, 69.02% yield), namely compound 7-2, as a brown solid. ESI-MS m/z: 455.10 [M+H]⁺.

### Step 3: Synthesis of Compound 7

Compound 7-2 (50 mg), (2-amino-3-chloro-6-fluoro-phenyl)boronic acid (8.33 mg) and Na₂CO₃ (9.32 mg) were dissolved in dioxane (1 mL) and acetonitrile (1 mL) at room temperature, under nitrogen protection, Pd(PPh₃)₄ (25.41 mg) was added into it, and the reaction solution was moved to 110° C. for 1 h. The reaction solution was poured into ethyl acetate, washed with an appropriate amount of water, the organic phase was dried over anhydrous sodium sulfate, filtered and concentrated. The concentrate was purified by silica gel reverse column chromatography and pre-TLC (EA/PE=2:3) to obtain the target product (4.0 mg, 19.19% yield, 95.01% purity), namely compound 7, as a pale yellow solid. ESI-MS m/z: 450.10 [M+H]⁺.

### Example 8: Compound 1-(3-(2-chloro-2′,6-difluoro-6′-hydroxy-[1,1′-biphenyl]-4-yl)-5,6-dihydroimidazo [1,5-a]pyrazin-7(8H)-yl)prop-2-en-1-one

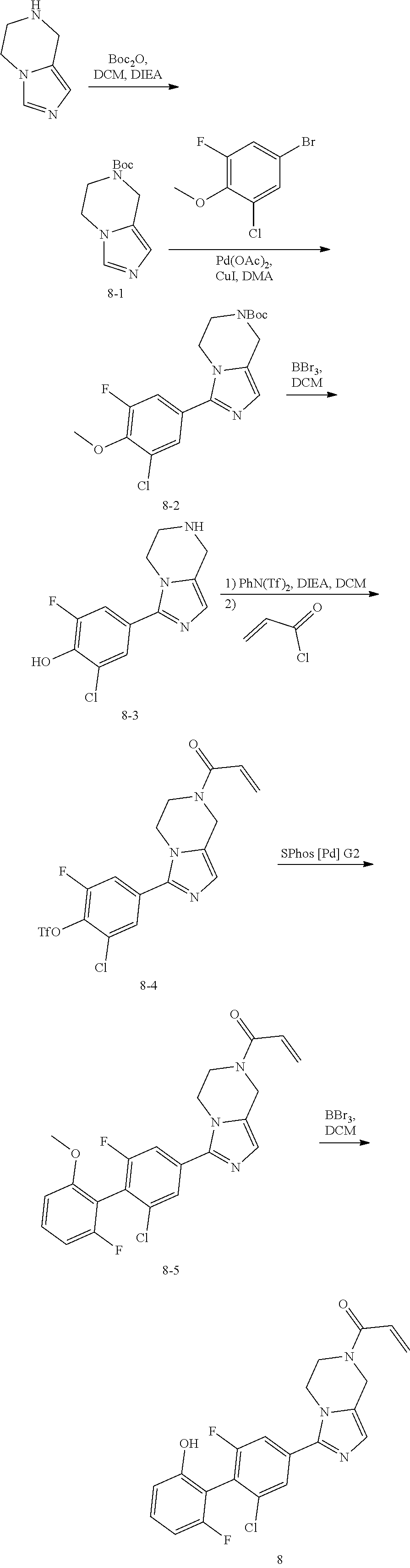

### Step 1: Synthesis of Compound 8-1

At room temperature, compound 5,6,7,8-tetrahydroimidazo[1,5-a]pyrazine (1 g) was dissolved in DCM, then DIEA (2.31 g) was added, and after cooling to 0° C., tert-butoxycarbonyl tert-butyl carbonate (1.95 g) dissolved in DCM was added slowly and the reaction was allowed to return to room temperature while stirring. After the reaction was completed, a small amount of water was added for washing, extracted with DCM (containing 5% methanol) for several times, the organic phase was dried and concentrated to obtain the target product (1.76 g, 97.08% yield) as colorless oil, namely compound 8-1. ESI-MS m/z: 224.11 [M+H]⁺.

### Step 2: Synthesis of Compound 8-2

Compound 8-1 (800 mg) and 5-bromo-1-chloro-3-fluoro-2-methoxybenzene (1.29 g) were dissolved in DMA (3 mL) at room temperature and under nitrogen iodinated cuprous (682.40 mg) and Pd(OAc)₂ (80.33 mg) were added, and the reaction solution was moved to 160° C. and reacted overnight. The reaction solution was cooled to room temperature, diluted with EA, washed with water several times, and the organic phase was dried and concentrated. The concentrate was purified by pre-TLC to obtain the target product (213 mg, 15.57% yield), namely compound 8-2. ¹H NMR (500 MHz, CDCl₃) δ: 7.45 (s, 1H), 7.34 (d, J=11.3 Hz, 1H), 6.98 (s, 1H), 4.72 (s, 2H), 4.12 (s, 2H), 4.02 (s, 3H), 3.79 (s, 2H), 1.51 (s, 9H). ESI-MS m/z: 382.12 [M+H]⁺.

### Step 3: Synthesis of Compound 8-3

The above compound 8-2 was dissolved in DCM (5 ml) at room temperature, then cooled to 0° C., BBr₃ (1.17 g) diluted with DCM (5 ml) was added, and the reaction was returned to room temperature. The system formed a lot of white solids and the system became cloudy. After the reaction was completed, it was cooled to 0° C., methanol was added to quench the reaction, and then N₂ was degassed, and then concentrated, the concentrate was compound 8-3, and the next reaction was directly carried out. ESI-MS m/z: 382.12 [M+H]⁺.

### Step 4: Synthesis of Compounds 8-4

The above compound 8-3 was dissolved in DCM at room temperature, then DIEA (194.09 mg) was added, then 1,1,1-trifluoro-N-phenyl-N-(trifluoromethylsulfonyl)methane sulfonic acid (295.08 mg) was added and stirred at room temperature. After LC-MS detected that the reaction of the raw materials was complete, the reaction solution was cooled to 0° C., and acryloyl chloride (67.96 mg) was added thereto, and the reaction was maintained at 0° C. After the reaction was completed, add sodium bicarbonate aqueous solution to it to quench, the reaction mixture was added EA to extract, combined the organic phases, dried and concentrated. The concentrate was purified by pre-TLC to obtain the target product (290 mg, 85.11% yield), namely compound 8-4. ESI-MS m/z: 454.11 [M+H]⁺.

### Step 5: Synthesis of Compounds 8-5

Compound 8-4 (150 mg), (2-fluoro-6-methoxyphenyl)boronic acid (61.79 mg) and K₃PO₄ (210.52 mg) were dissolved in dioxane (6 mL) and H₂O (0.5 mL) at room temperature. In the mixed solvent, under nitrogen protection atmosphere, Sphos Pd G2 (23.80 mg) was added to it, and then moved to microwave at 85° C. for 35 min. The reaction solution was cooled to room temperature, diluted with EA, washed with water, and the organic phases were combined, dried, and concentrated. The concentrate was purified by pre-TLC to give the target product (135 mg, 95.01% yield) as colorless oil, namely compound 8-5. ESI-MS m/z: 430.11 [M+H]⁺.

### Step 6: Synthesis of Compound 8

Compound 8-5 (62 mg) was dissolved in DCM (2 ml) at room temperature, then the reaction solution was cooled to 0° C., BBr₃ (361.35 mg) diluted with DCM (1 ml) was added, and then moved to room temperature to react for about 30 min. The reaction was quenched with methanol at 0° C. and concentrated. The concentrate was then dissolved in THF, a small amount of NaOH solution was added, after 5 min, THF was removed, a small amount of water was added, and then EA was extracted for several times, the organic phases were combined, dried, and concentrated. The concentrate was purified by pre-TLC to give the target product (27.6 mg, 46.02% yield, 99.20% purity), compound 8, as a white solid. ESI-MS m/z: 416.11 [M+H]⁺.

### Example 9: Compound 1-(3-(2,3′-dichloro-6,6′-difluoro-2′-hydroxy-[1,1′-biphenyl]-4-yl)-5,6-dihydroimidazo[1,5-a]pyrazin-7(8H)-yl)prop-2-en-1-one and/or 1-(3-(2,3′-dichloro-2′,6-difluoro-6′-hydroxy-[1,1′-biphenyl]-4-yl)-5,6-dihydroimidazo[1,5-a]pyrazin-7(8H)-yl) prop-2-en-1-one

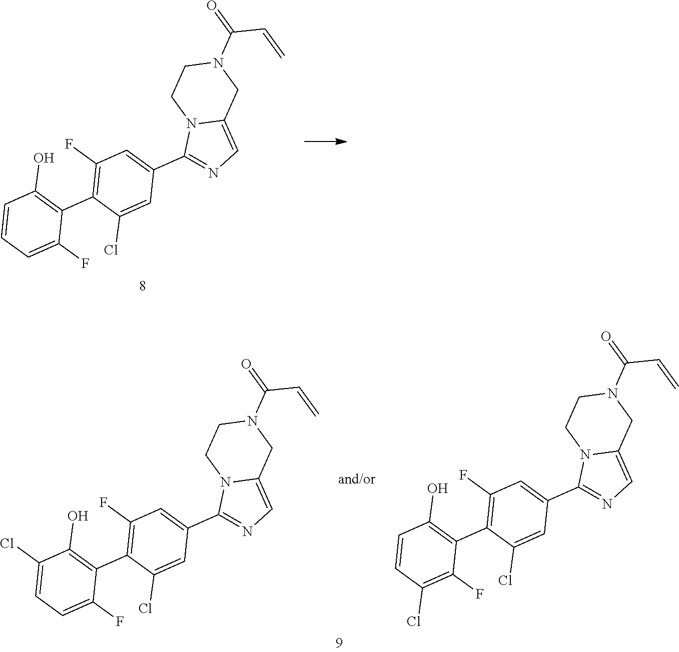 [SMILES: C=CC(=O)N1CCn2c(cnc2-c2cc(F)c(-c3c(O)cccc3F)c(Cl)c2)C1 ; C=CC(=O)N1CCn2c(cnc2-c2cc(F)c(-c3c(F)ccc(Cl)c3O)c(Cl)c2)C1 ; C=CC(=O)N1CCn2c(cnc2-c2cc(F)c(-c3c(O)ccc(Cl)c3F)c(Cl)c2)C1]

Compound 8 (38 mg) was dissolved in acetonitrile (3 mL) at room temperature, DIEA (11.81 mg) was added, and then an acetonitrile solution of 1-chloropyrrolidine-2,5-dione (24.41 mg) was added dropwise, the reaction was stirred at room temperature for 1 h. EA and water were directly added to the reaction solution to dilute, then extracted three times, the organic phases were combined, dried and concentrated. The concentrate was purified by pre-HPLC to give the target product (5.8 mg, 14.1% yield, 97.70% purity), compound 9, as a white solid. ESI-MS m/z: 450.11 [M+H]⁺.

### Example 10: Compound 1-(3-(2,3′,5′-trichloro-2′,6-difluoro-6′-hydroxy-[1,1′-biphenyl]-4-yl)-5,6-dihydroimidazo[1,5-a]pyrazin-7(8H)-yl)prop-2-en-1-one

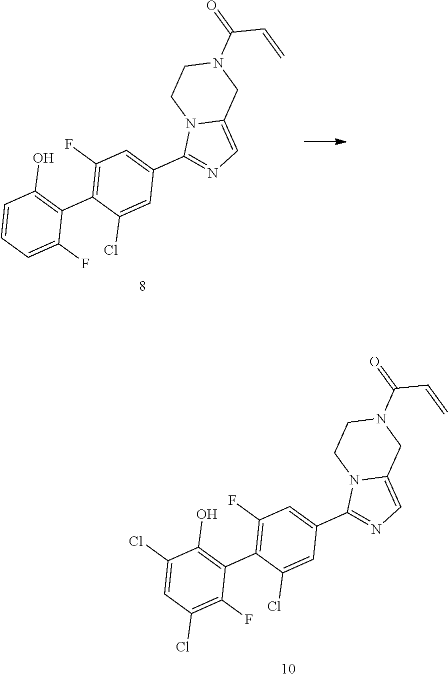 [SMILES: C=CC(=O)N1CCn2c(cnc2-c2cc(F)c(-c3c(O)cccc3F)c(Cl)c2)C1 ; C=CC(=O)N1CCn2c(cnc2-c2cc(F)c(-c3c(O)c(Cl)cc(Cl)c3F)c(Cl)c2)C1]

The steps are the same as the Example 9. The concentrate was purified by pre-HPLC to give the target product (5.8 mg, 13.9% yield, 97.50% purity), compound 10, as a white solid. ESI-MS m/z: 484.12 [M+H]⁺.

### Example 11: Compound 1-(3-(2,2′-difluoro-6′-hydroxy-6-vinyl-[1,1′-biphenyl]-4-yl)-5,6-dihydro-[1,2,4]triazolo[4,3-a]pyrazin-7(8H)-yl)prop-2-en-1-one

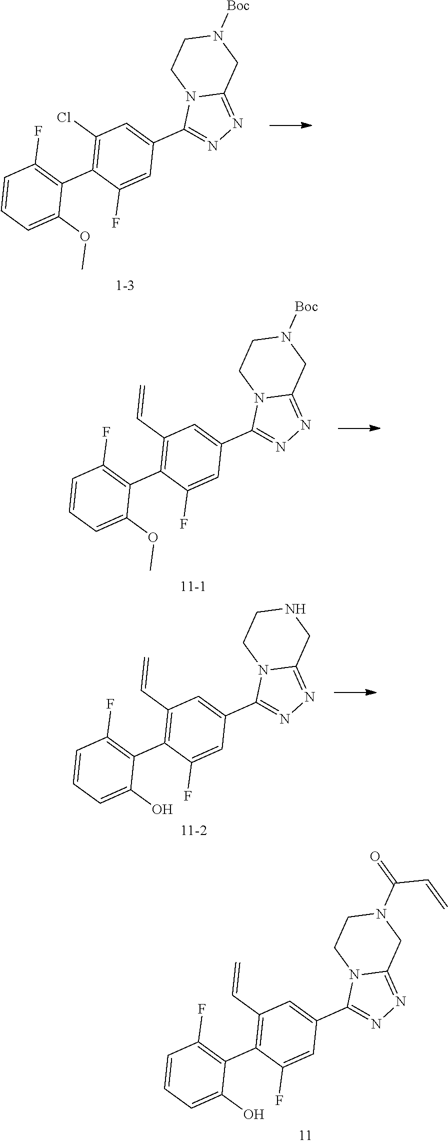 [SMILES: COc1cccc(F)c1-c1c(F)cc(-c2nnc3n2CCN(C(=O)OC(C)(C)C)C3)cc1Cl ; C=Cc1cc(-c2nnc3n2CCN(C(=O)OC(C)(C)C)C3)cc(F)c1-c1c(F)cccc1OC ; C=Cc1cc(-c2nnc3n2CCNC3)cc(F)c1-c1c(O)cccc1F ; C=CC(=O)N1CCn2c(nnc2-c2cc(F)c(-c3c(O)cccc3F)c(C=C)c2)C1]

### Step 1: Synthesis of Compound 11-1

Compound 1-3 (46 mg), 4,4,5,5-tetramethyl-2-vinyl-1,3,2-dioxaborane (29.7 mg) were combined with K₃PO₄ (61.42 mg) at room temperature was dissolved in a mixed solvent of dioxane (2 mL) and H₂O (0.5 mL), under nitrogen protection, Sphos Pd G2 (6.9 mg) was added to it, and the reaction solution was moved to 100° C. and stirred for 4 h. The reaction solution was concentrated, diluted with a small amount of DCM, and purified by pre-TLC to obtain the target product (22 mg, 48.8% yield) as a white solid, namely compound 11-1. ESI-MS m/z: 469.4 [M+H]⁺.

### Step 2: Synthesis of Compound 11-2

At room temperature, compound 11-1 (22 mg) was dissolved in DCM (2 mL), and BBr₃ (58.82 mg) was slowly added dropwise in an ice-water bath. The reaction solution was poured into an ice-water bath, methanol was slowly added to quench the reaction, and concentrated. The concentrate was purified by pre-TLC to obtain the target product (10 mg, 60.2% yield), compound 11-2, as a white solid. ESI-MS m/z: 355.4 [M+H]⁺.

### Step 3: Synthesis of Compound 11

Compound 11-2 (10 mg) was dissolved in DCM (2 mL) at room temperature, DIEA (10.94 mg) was added, the reaction solution was cooled to 0° C., and a solution of acryloyl chloride (2.55 mg) diluted with DCM was added to it, and the reaction was kept at 0° C. for 1 h. The reaction solution was directly concentrated, and the concentrate was purified by pre-TLC to obtain the target product (3.3 mg, 27.53% yield, 96.15% purity), namely compound 11, as a white solid. ESI-MS m/z: 409.25 [M+H]⁺.

### Example 12: Compound 1-(3-(2-Chloro-2′,6-difluoro-6′-hydroxy-[1,1′-biphenyl]-4-yl)-2-(2-isopropyl phenyl)-5,6-dihydroimidazo[1,2-a]pyrazin-7(8H)-yl)prop-2-en-1-one

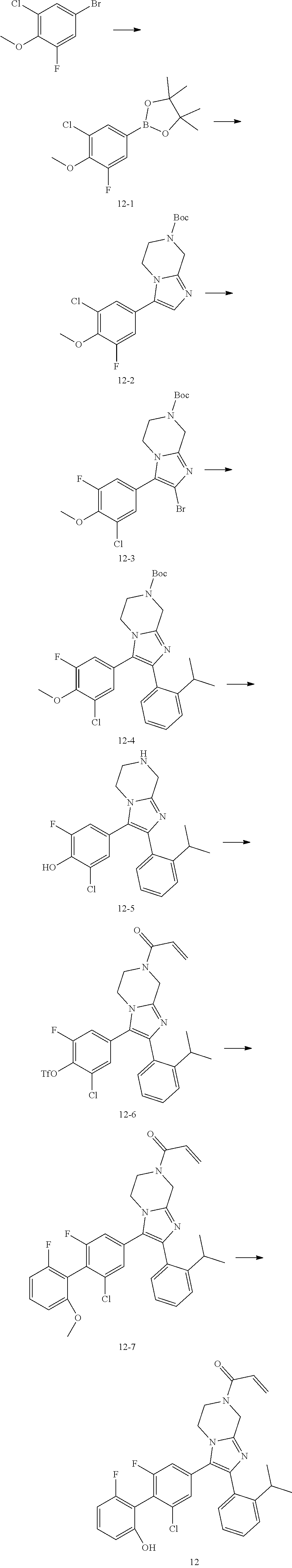

### Step 1: Synthesis of Compound 12-1

At room temperature, compound 5-bromo-1-chloro-3-fluoro-2-methoxybenzene (8.7 g), 4,4,5,5-tetramethyl-2-(4,4,5,5-tetramethyl-1,3,2-dioxaboran-2-yl)-1,3,2-dioxaborane (13.84 g) and KOAc (10.68 g) were dissolved in dioxane (87 mL). In the nitrogen protection atmosphere, Pd(dppf)Cl₂ (2.96 g) was added into it, and the reaction mixture was moved to 80° C. for reaction for 2 h. The reaction solution was diluted with water, extracted with EA three times, the organic phases were combined, dried over anhydrous sodium sulfate, filtered and concentrated. The concentrate was purified by silica gel column chromatography (PE:DCM=10:1) to obtain the target product (7.75 g, 74.45% yield), compound 12-1, as a colorless oil.

### Step 2: Synthesis of Compound 12-2

At room temperature, compound 12-1 (5.8 g), tert-butyl 3-bromo-6,8-dihydro-5H-imidazo[1,2-a]pyrazine-7-carboxylate (5.50 g) and Cs₂CO₃ (19.78 g) were dissolved in a mixed solvent of dioxane (120 mL) and H₂O (30 mL), under nitrogen protection atmosphere, add Pd(dppf)Cl₂ (1.65 g) into it, move the reaction mixture to 90° C. and react for 2 h. The reaction solution was diluted with water, extracted with EA three times, the organic phases were combined, dried over anhydrous sodium sulfate, filtered and concentrated. The concentrate was purified by silica gel column chromatography (DCM:MeOH=30:1) to obtain the target product (6 g, 77.63% yield), compound 12-2, as a yellow foam.

### Step 3: Synthesis of Compound 12-3

Compound 12-2 (6 g) and 1-bromopyrrolidine-2,5-dione (4.20 g) were dissolved in acetonitrile (60 mL) at room temperature, and the reaction was stirred at room temperature for 15 min. The reaction solution was diluted with water, extracted with EA three times, the organic phases were combined, dried over anhydrous sodium sulfate, filtered and concentrated. The concentrate was purified by silica gel column chromatography (PE:EA=2:1) to obtain the yellow foamy target product (6 g, 77.63% yield), namely compound 12-3.

### Step 4: Synthesis of Compound 12-4

Compound 12-3 (630 mg), (2-isopropylphenyl)boronic acid (448.54 mg) and K₂CO₃ (471.76 mg) were dissolved in a mixed solvent of H₂O (1 mL) and dioxane (12 mL) at room temperature under nitrogen. In a protective atmosphere, Pd(dppf)Cl₂.DCM (111.58 mg) was added, and the reaction mixture was moved to 110° C. for 12 h. The reaction solution was diluted with water, extracted with EA three times, the organic phases were combined, dried over anhydrous sodium sulfate, filtered and concentrated. The concentrate was purified by silica gel column chromatography (PE:EA=0-20%) to obtain the target product (260 mg, 38.03% yield), compound 12-4, as a colorless oil. ESI-MS m/z: 500.24 [M+H]⁺.

### Step 5: Synthesis of Compound 12-5

At room temperature, compound 12-4 (250 mg) was dissolved in DCM (5 mL), and the temperature was lowered to 0° C., and then a solution of BBr₃ (626.30 mg) in DCM (2 mL) was added to the reaction solution. After the addition, the temperature was slowly raised to room temperature for 1 h. The reaction was quenched by adding methanol at low temperature (under −40° C.), and then the solvent was spin-dried to obtain the crude target product (260 mg) as a yellow oil, namely compound 12-5. ESI-MS m/z: 386.23 [M+H]⁺.

### Step 6: Synthesis of Compound 12-6

At room temperature, compound 12-5 (190 mg) was dissolved in DCM (6 mL), DIEA (318.20 mg) was added, and the reaction solution was clarified, followed by N-phenylbis(trifluoromethanesulfonimide) (193.37 mg), reacted at room temperature for 0.5 h, LC-MS detection showed that the reaction was completed, it was cooled to 0° C., acryloyl chloride (49.02 mg) was added to it, and the reaction was maintained at 0° C. for 0.2 h. The reaction solution was diluted with water, extracted three times with DCM, the organic phases were combined, dried over anhydrous sodium sulfate, filtered and concentrated. The concentrate was purified by silica gel column chromatography (DCM:MeOH=100:3) to obtain the target product (190 mg, 67.46% yield), compound 12-6, as a yellow solid. ESI-MS m/z: 518.12 [M+H]⁺.

### Step 7: Synthesis of Compound 12-7

Compound 12-6 (150 mg), (2-fluoro-6-methoxy-phenyl)boronic acid (89.14 mg) and K₃PO₄ (138.99 mg) were dissolved in H₂O (0.2 mL) and dioxane (3 mL) at room temperature. In the mixed solvent, under a nitrogen atmosphere, Sphos-Pd-G2 (19.19 mg) was added, and the temperature of the reaction solution was raised to 100° C. for 1.5 h. The reaction solution was diluted with water, extracted with EA three times, the organic phases were combined, dried over anhydrous sodium sulfate, filtered and concentrated. The concentrate was purified by pre-TLC (DCM:MeOH:aqueous ammonia=18:1:0.18) to obtain the target product (110 mg, 76.54% yield) as a yellow oil, namely compound 12-7. ESI-MS m/z: 572.18 [M+H]⁺.

### Step 8: Synthesis of Compound 12

At room temperature, compound 12-7 (60 mg) was dissolved in dry DCM (2 mL), the temperature was lowered to 0° C., and a solution of BBr₃ (274.28 mg) in DCM (1 mL) was added to the reaction solution, and the addition was completed and returned to room temperature reaction for 1 h. Methanol was add at low temperature to quench the reaction, then spin the solvent to dry, add 2 mL of THF and 0.5 mL of water, then add a small amount of NaOH aqueous solution, react at room temperature for 0.2 h. the reaction solution was detected that all hydrogen bromide was eliminated, then add water and EA for extraction, the reaction solution was dried and concentrated. The concentrate was purified by pre-TLC (DCM:MeOH:ammonia=15:1:0.15) to give the target product (25 mg, 42.76% yield), compound 12, as a white solid. ESI-MS m/z: 548.21 [M+H]⁺.

### Example 13: Compound 1-(3-(2,3′-dichloro-6,6′-difluoro-2′-hydroxy-[1,1′-biphenyl]-4-yl)-2-(2-isopropylphenyl)-5,6-dihydroimidazo[1,2-a]pyrazin-7(8H)-yl)prop-2-en-1-one and/or 1-(3-(2,3′-dichloro-2′,6-difluoro-6′-hydroxy-[1,1′-biphenyl]-4-yl)-2-(2-isopropylphenyl)-5,6-dihydroimidazo[1,2-a]pyrazin-7(8H)-yl)prop-2-en-1-one

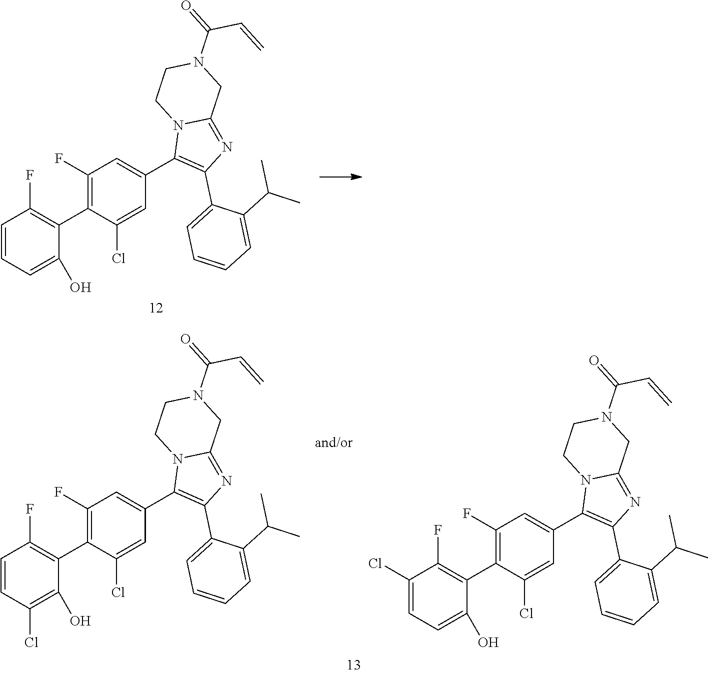 [SMILES: C=CC(=O)N1CCn2c(nc(-c3ccccc3C(C)C)c2-c2cc(F)c(-c3c(O)cccc3F)c(Cl)c2)C1 ; C=CC(=O)N1CCn2c(nc(-c3ccccc3C(C)C)c2-c2cc(F)c(-c3c(F)ccc(Cl)c3O)c(Cl)c2)C1 ; C=CC(=O)N1CCn2c(nc(-c3ccccc3C(C)C)c2-c2cc(F)c(-c3c(O)ccc(Cl)c3F)c(Cl)c2)C1]

At room temperature, compound 12 (20 mg) and 1-chloropyrrolidine-2,5-dione (10.00 mg) were dissolved in acetonitrile (0.6 mL) and reacted at room temperature for 5 h. The reaction solution was diluted with water, extracted with EA three times, the organic phases were combined, dried over anhydrous sodium sulfate, filtered and concentrated. The concentrate was purified by pre-HPLC to give the target product (2.5 mg, 7.83% yield, 98.12% purity) compound 13, as a white solid. ESI-MS m/z: 568.16 [M+H]⁺.

### Example 14: Compound 1-(2-(2-isopropylphenyl)-3-(2,3′,5′-trichloro-2′,6-difluoro-6′-hydroxy-[1, 1′-biphenyl]-4-yl)-5,6-dihydroimidazo[1,2-a]pyrazin-7(8H)-yl)prop-2-en-1-one

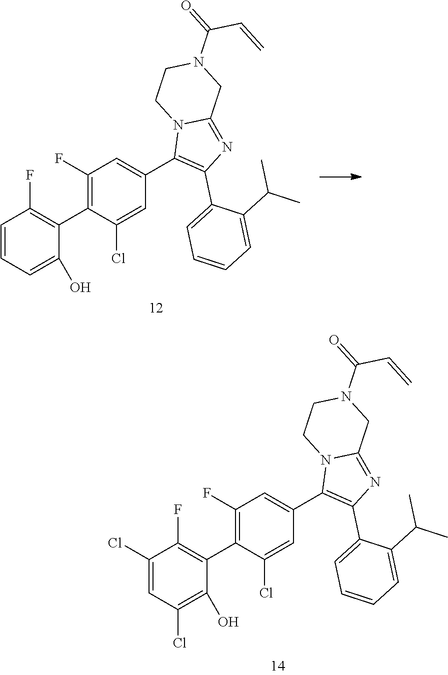 [SMILES: C=CC(=O)N1CCn2c(nc(-c3ccccc3C(C)C)c2-c2cc(F)c(-c3c(O)cccc3F)c(Cl)c2)C1 ; C=CC(=O)N1CCn2c(nc(-c3ccccc3C(C)C)c2-c2cc(F)c(-c3c(O)c(Cl)cc(Cl)c3F)c(Cl)c2)C1]

The procedure was the same as above, and the concentrate was purified by pre-HPLC to obtain the target product as a white solid (8.1 mg, 23.91% yield, 97.55%), namely compound 14. ESI-MS m/z: 602.16[M+H]⁺.

### Example 15: Compound 1-(3-(2,3′-Dichloro-6,6′-difluoro-2′-hydroxy-[1,1′-biphenyl]-4-yl)-5,6-dihydroimidazo[1,2-a]pyrazin-7(8H)-yl)prop-2-en-1-one

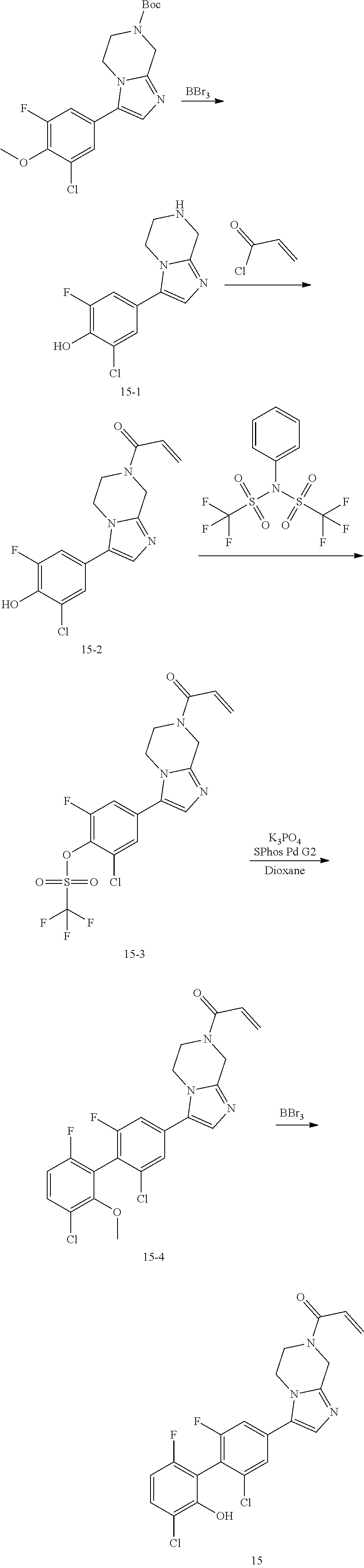

### Step 1: Synthesis of Compound 15-1

At room temperature, compound 3-(3-chloro-5-fluoro-4-methoxy-phenyl)-6,8-dihydro-5H-imidazo[1,2-a]pyrazine-7-tert-butyl carboxylate (300 mg) was dissolved in DCM (15 mL), and after cooling in an ice-water bath, BBr₃ (590.50 mg) was slowly added dropwise, followed by stirring at room temperature for 10 min. The reaction was quenched by slowly adding methanol dropwise under an ice-water bath and concentrated. The concentrate was dissolved in ether and recrystallized to obtain the target product (200 mg, 95.09% yield) as a yellow solid, namely compound 15-1. ESI-MS m/z: 268.06 [M+H]⁺.

### Step 2: Synthesis of Compound 15-2

Compound 15-1 (200 mg) and DIEA (193.13 mg) were dissolved in DCM (9 mL) at room temperature. After cooling in an ice-water bath, diluted acryloyl chloride (67.62 mg) was slowly added dropwise, and the reaction was performed in an ice-water bath for 10 min. The reaction solution was directly concentrated to obtain the target product (230 mg, 95.68% yield) as a pale yellow solid, namely compound 15-2. ESI-MS m/z: 322.21 [M+H]⁺.

### Step 3: Synthesis of Compound 15-3

Compound 15-2 (230 mg), 1,1,1-trifluoro-N-phenyl-N-(trifluoromethylsulfonyl) methanesulfonic acid (383.08 mg) and DIEA (184.79 mg) were combined at room temperature The mixture was dissolved in DCM (9 mL) and stirred at room temperature for 30 min. The reaction solution was directly concentrated, and the concentrate was purified by pre-TLC (EA) to obtain the target product (125 mg, 38.53% yield) as a white solid, namely compound 15-3. ESI-MS m/z: 454.11 [M+H]⁺.

### Step 4: Synthesis of Compound 15-4

Compound 15-3 (90 mg), (3-chloro-6-fluoro-2-methoxy-phenyl)boronic acid (81.07 mg), K₃PO₄ (63.15 mg) and SPhos Pd G2 (14.27 mg) were mixed at room temperature. It was mixed in dioxane (15 mL), and heated to 90° C. by microwave under nitrogen protection for 80 min. The insoluble matter was removed by filtration with Celite, and the filtrate was concentrated. The concentrate was purified by pre-TLC (EA:MeOH=10:1) to obtain the target product (35 mg, 38.04% yield), compound 15-4, as a pale yellow solid. ESI-MS m/z: 464.21 [M+H]⁺.

### Step 5: Synthesis of Compound 15

At room temperature, compound 15-4 (35 mg) was dissolved in DCM (6 mL), cooled in an ice-water bath, BBr₃ (37.77 mg) was slowly added dropwise, and the reaction was stirred at room temperature for 10 min. In an ice-water bath, methanol was used to quench the reaction, concentrated, the concentrate was dissolved in THF/H₂O (4/1), 4N NaOH solution was added dropwise, stirred at room temperature for 5 min, and then concentrated. The concentrate was purified by pre-HPLC to give the target product (1 mg, 2.95% yield, 95% purity), compound 15, as a white solid. ESI-MS m/z: 450.12 [M+H]⁺.

### Example 16: Compound 1-(3-(2,3′-dichloro-6′-fluoro-2′-hydroxy-6-(2,2,2-trifluorooxy)-[1,1′-biphenyl]-4-yl)-5,6-dihydro-[1,2,4]triazolo[4,3-a]pyrazin-7(8H)-yl)prop-2-en-1-one

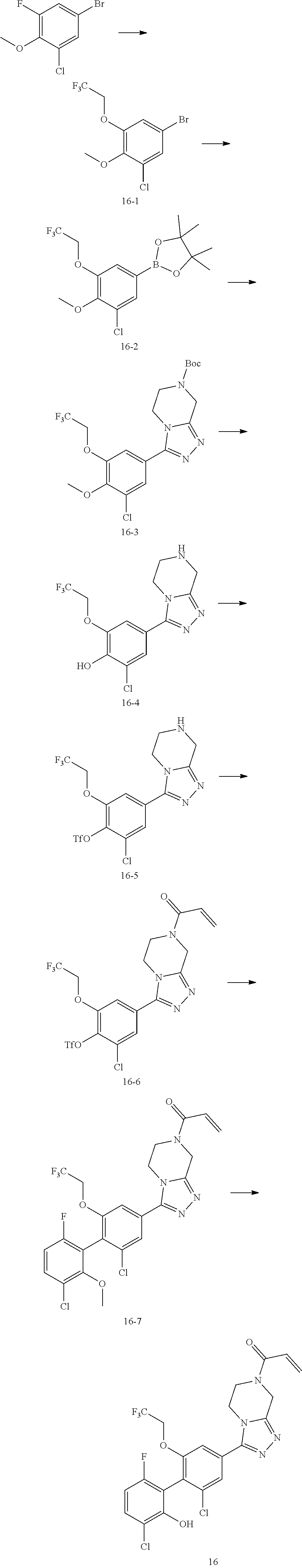

### Step 1: Synthesis of Compound 16-1

Compound 5-bromo-1-chloro-3-fluoro-2-methoxybenzene (1 g) was dissolved in DMF (10 mL) at room temperature, then trifluoroethanol (835.51 mg) and sodium tert-butoxide (802.64 mg) were added to it, and the reaction was carried out at 100° C. for 1 h. The reaction solution was diluted with water, extracted with EA three times, the organic phases were combined, dried over anhydrous sodium sulfate, filtered and concentrated. The concentrate was purified by silica gel column chromatography (PE:EA=0-2%) to obtain the target product (100 mg, 67.46% yield), compound 16-1, as a colorless liquid.

### Step 2: Synthesis of Compound 16-2

At room temperature, compound 16-1 (900 mg), 4,4,5,5-tetramethyl-2-(4,4,5,5-tetramethyl-1,3,2-dioxol-2-yl)-1,3,2-dioxol (715.31 mg) and CH₃COOK (276.05 mg) were dissolved in dioxane (15 mL), under a nitrogen atmosphere, and Pd(dppf)Cl₂.DCM (2.30 g) was added and the reaction was moved to 100° C. and stirred for 2 h. The reaction solution was diluted with water, extracted with EA three times, the organic phases were combined, dried over anhydrous sodium sulfate, filtered and concentrated. The concentrate was purified by silica gel column chromatography (PE:EA=0-5%) to obtain the target product (800 mg, 77.48% yield), compound 16-2, as a colorless liquid.

### Step 3: Synthesis of Compound 16-3

At room temperature, compound 16-2 (700 mg, 1.91 mmol), 3-bromo-6,8-dihydro-5H-[1,2,4]triazolo[4,3-a]pyrazine-7-tert-butyl carboxylate (578.91 mg) and Cs₂CO₃ (1.24 g) were dissolved in a mixed solvent of dioxane (12 mL) and H₂O (1 mL), under a nitrogen atmosphere, and Pd(dppf)Cl₂.DCM (155.82 mg) was added, and the reaction was raised to 90° C. for 0.8 h. The reaction solution was diluted with water, extracted with EA three times, the organic phases were combined, dried over anhydrous sodium sulfate, filtered and concentrated. The concentrate was purified by silica gel column chromatography (DCM:MeOH=0-3%) to obtain the target product (700 mg, 79.20% yield), compound 16-3, as a yellow solid. ESI-MS m/z: 463.21 [M+H]⁺.

### Step 4: Synthesis of Compound 16-4

At room temperature, compound 16-3 (650 mg) was dissolved in dry DCM (15 mL), cooled to 0° C., and then BBr₃ (1.76 g) in DCM (3 mL) solution was slowly added to the reaction solution, then return to room temperature for 1.5 h. The reaction solution was cooled to −20° C., methanol was slowly added dropwise to quench the reaction, and then concentrated to obtain the target product (680 mg) as a yellow liquid, namely compound 16-4. ESI-MS m/z: 349.13 [M+H]⁺.

### Step 5: Synthesis of Compound 16-5

Compound 16-4 (488.19 mg) was dissolved in DCM (15 mL) at room temperature, DIEA (904.70 mg) was added dropwise, and then N-phenylbis(trifluoromethanesulfonimide) (649.74 mg) was added dropwise and reacted at room temperature for 1 h. The target product was compound 16-5. ESI-MS m/z: 481.08 [M+H]⁺.

### Step 6: Synthesis of Compound 16-6

The reaction solution of the above compound 16-5 was cooled to 0° C. and a solution of acryloyl chloride (152.04 mg) in DCM (1 mL) was added to the reaction solution for 0.5 h. The reaction solution was diluted with water, extracted three times with DCM, the organic phases were combined, dried over anhydrous sodium sulfate, filtered and concentrated. The concentrate was purified by silica gel column chromatography (DCM:MeOH=0-3%) to obtain the target product (280 mg, 37.40% yield), compound 16-6, as a yellow solid. ESI-MS m/z: 535.07 [M+H]⁺.

### Step 7: Synthesis of Compound 16-7

Compound 16-6 (150 mg), (3-chloro-6-fluoro-2-methoxy-phenyl)boronic acid (57.33 mg) and K₃PO₄ (118.92 mg) were dissolved in H₂O (0.3 mL) at room temperature. In a mixed solvent with dioxane (3 mL), under nitrogen atmosphere, Sphos-Pd-G2 (20.50 mg) was added, and the reaction temperature was raised to 100° C. for 4 h. The reaction solution was diluted with water, extracted with EA three times, the organic phases were combined, dried over anhydrous sodium sulfate, filtered and concentrated. The concentrate was purified by pre-TLC (DCM:MeOH:aqueous ammonia=20:1:0.2) to obtain the target product (35 mg, 22.88% yield), compound 16-7, as a yellow solid. ESI-MS m/z: 545.11 [M+H]⁺.

### Step 8: Synthesis of Compound 16

At room temperature, compound 16-7 (35 mg) was dissolved in DCM (2 mL), the temperature of the reaction solution was cooled to 0° C., BBr₃ (80.40 mg) was added, and after the dropwise addition, the reaction was returned to room temperature for 1 h. Under an ice-water bath, the reaction was quenched by adding ice-water, then extracted with EA, dried and concentrated. The concentrate was purified by pre-TLC (DCM:MeOH=10:1) to give the desired product (11.3 mg, 33.14% yield, 98.64% purity), compound 16, as a pale yellow solid. ESI-MS m/z: 531.13 [M+H]⁺.

### Example 17: Compound

### 1-(3-(2-Chloro-2′-fluoro-6′-hydroxy-6-(2,2,2-trifluorooxy)-[1,1′-biphenyl]-4-yl)-5,6-dihydro-[1,2,4]triazolo[4,3-a]pyrazin-7(8H)-yl)prop-2-en-1-one

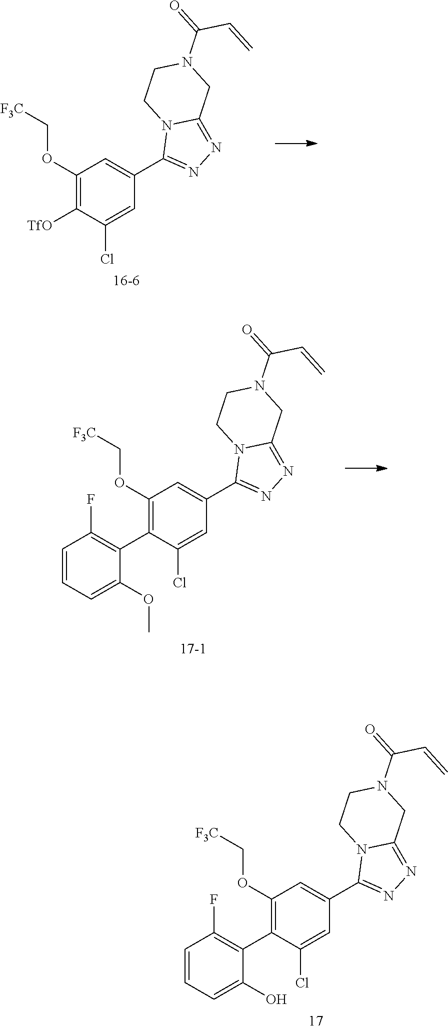 [SMILES: C=CC(=O)N1CCn2c(nnc2-c2cc(Cl)c(OS(=O)(=O)C(F)(F)F)c(OCC(F)(F)F)c2)C1 ; C=CC(=O)N1CCn2c(nnc2-c2cc(Cl)c(-c3c(F)cccc3OC)c(OCC(F)(F)F)c2)C1 ; C=CC(=O)N1CCn2c(nnc2-c2cc(Cl)c(-c3c(O)cccc3F)c(OCC(F)(F)F)c2)C1]

### Step 1: Synthesis of Compound 17-1

Compound 16-6 (80 mg), (2-fluoro-6-methoxyphenyl)boronic acid (38.13 mg) and K₃PO₄ (95.25 mg) were dissolved in a solution of dioxane (5 mL) and H₂O (0.5 mL) at room temperature, in the mixed solvent, under a nitrogen atmosphere, Sphos Pd G2 (10.77 mg) was added, and the reaction was carried out at 100° C. for 4 h. The reaction solution was cooled to room temperature, diluted with water, extracted three times with EA, the organic phases were combined, dried over anhydrous sodium sulfate, filtered and concentrated. The concentrate was purified by pre-TLC (DCM:MeOH=20:1) to give the target product (40 mg, 52.34% yield), compound 17-1, as a yellow solid. ESI-MS m/z: 511.17 [M+H]⁺.

### Step 2: Synthesis of Compound 17

At room temperature, compound 17-1 (40 mg) was dissolved in dry DCM (5 mL), and BBr₃ (195.75 mg) was added dropwise in an ice-water bath. After the addition was completed, the ice bath was removed, and the reaction was naturally raised to room temperature for 30 min. Under an ice-water bath, the reaction was quenched with ice-water, extracted three times with EA, and the organic phases were combined, dried over anhydrous sodium sulfate, filtered, and concentrated. The concentrate was purified by pre-TLC (DCM:MeOH=20:1) to give the target product (14.2 mg, 34.84% yield, 95.44% purity), compound 17, as a white solid. ESI-MS m/z: 497.27 [M+H]⁺.

### Example 18: Compound 1-(3-(2,3′-Dichloro-6,6′-difluoro-2′-hydroxy-[1,1′-biphenyl]-4-yl)-2-(trifluoromethyl)-5,6-di hydroimidazo[1,2-a]pyrazin-7(8H)-yl)prop-2-en-1-one

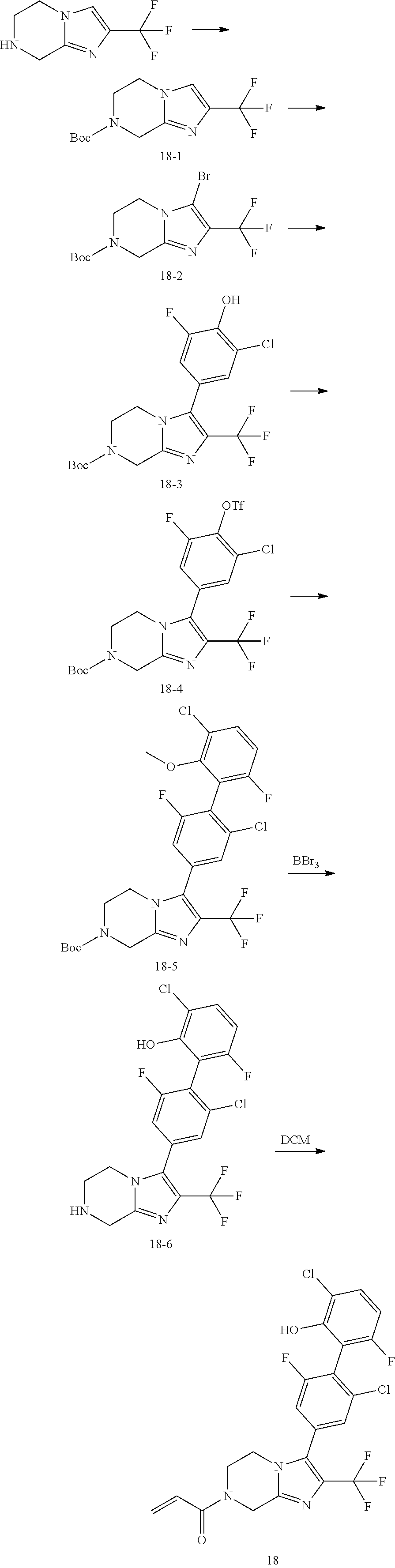

### Step 1: Synthesis of Compound 18-1

At 0° C., Compound 2-(trifluoromethyl)-5,6,7,8-tetrahydroimidazo[1,2-a]pyrazine (1 g) and DIEA (2.03 g) were dissolved in DCM (10 mL), then tert-butoxycarbonyl tert-butyl carbonate (1.71 g) was added, and stirred at room temperature for 2 h. DCM was added to the reaction solution, the organic phase was washed with water, dried over anhydrous sodium sulfate, filtered and concentrated. The target product (1.3 g), compound 18-1, was obtained as a pale yellow solid. ESI-MS m/z: 292.00 [M+H]⁺.

### Step 2: Synthesis of Compound 18-2

At room temperature, compound 18-1 (1.2 g) was dissolved in acetonitrile (10 mL), 1-bromopyrrolidine-2,5-dione (953.26 mg) was added at 0° C., and then slowly returned to room temperature and stirred 1 h. The reaction solution was poured into water, extracted three times with ethyl acetate, the organic phases were combined, dried over anhydrous sodium sulfate, filtered and concentrated. The concentrate was purified by reverse-phase silica gel column chromatography (CH₃CN:H₂O=0-95%) to obtain the target product (1.21 g, 78.69% yield), compound 18-2, as a white solid. ESI-MS m/z: 370.09 [M+H]⁺.

### Step 3: Synthesis of Compound 18-3

Compound 18-2 (1 g), (3-chloro-5-fluoro-4-hydroxyphenyl)boronic acid (514.27 mg) and Cs₂CO₃ (1.76 g) were dissolved in dioxane (6 mL) and H₂O (0.5 mL) at room temperature, in a nitrogen atmosphere, Pd(dppf)Cl₂ (197.48 mg) was added to it, and the reaction was carried out at 85° C. for 35 min by microwave. Ethyl acetate was added to the reaction solution, and the organic phase was washed with water, dried over anhydrous sodium sulfate, filtered, and concentrated to obtain the target product (950 mg) as a black solid, namely compound 18-3. ESI-MS m/z: 436.16 [M+H]⁺.

### Step 4: Synthesis of Compound 18-4

Compound 18-3 (800 mg, 1.84 mmol) and DIEA (711.76 mg) were dissolved in DCM (20 mL) at room temperature, and then 1,1,1-trifluoro-N-phenyl-N-(trifluoromethylsulfonyl) methanesulfonic acid (983.71 mg) and stirring was continued for 1 h. Ethyl acetate was added to the reaction solution, and the organic phase was washed with water, dried over anhydrous sodium sulfate, filtered, and concentrated. The concentrate was purified by silica gel column chromatography (PE:EA=0-30%) to obtain the target product (660 mg, 63.31% yield), compound 18-4, as a white solid. ¹H NMR (500 MHz, CDCl₃) δ: 7.33 (t, J=1.7 Hz, 1H), 7.22 (dd, J=9.3, 2.0 Hz, 1H), 3.93-3.81 (m, 6H), 1.50 (s, 9H). ESI-MS m/z: 568.08 [M+H]⁺.

### Step 5: Synthesis of Compound 18-5

Compound 18-4 (200 mg), (3-chloro-6-fluoro-2-methoxy-phenyl)boronic acid (71.99 mg) and K₃PO₄ (149.52 mg) were dissolved in dioxane (2 mL) and H₂O at room temperature (0.1 mL) of the mixed solvent, in a nitrogen atmosphere, Sphos Pd G2 (25.34 mg) was added to it, and the reaction was microwaved at 85° C. for 1.5 h. The reaction solution was directly concentrated, and the concentrate was purified by pre-TLC (PE:EA:DCM=1:0.2:1) to obtain the target product (48 mg, 23.56% yield) as a white solid, namely compound 18-5. ESI-MS m/z: 578.20 [M+H]⁺.

### Step 6: Synthesis of Compound 18-6

At room temperature, compound 18-5 (48 mg) was dissolved in DCM (15 mL), and BBr₃ (518.71 mg) was slowly added dropwise at 0° C. After the dropwise addition was completed, the mixture was slowly warmed to room temperature, and the reaction was stirred for 9 h. 2 ml of ice water was added to the reaction solution, followed by 10 ml of saturated sodium bicarbonate solution, extracted once with DCM, and then twice with ethyl acetate. The organic phase was dried over anhydrous sodium sulfate, filtered, and concentrated. The concentrate was purified by pre-TLC (DCM:MeOH=15:1) to give the target product (30 mg, 77.87% yield), compound 18-6, as a white solid. ESI-MS m/z: 464.16 [M+H]⁺.

### Step 7: Synthesis of Compound 18

Compound 18-6 (30 mg) was dissolved in DCM (5 mL) at room temperature, then acryloyl chloride (5.85 mg) was added with stirring at 0° C., and the reaction was continued to stir for 5 min. An appropriate amount of ice water was added to the reaction solution, and then DCM was added for extraction. The organic phase was dried over anhydrous sodium sulfate, filtered and concentrated. The concentrate was purified by pre-TLC (DCM:MeOH=15:1) to give the desired product (9.0 mg, 26.14% yield, 97.27% purity), compound 18, as a pale yellow solid. ESI-MS m/z: 518.09 [M+H]⁺.

### Biological Assay

### Example 1: Cell Proliferation Assay

Pave the MIA-PACA-2 cells into a 96-well ultra-low adsorption plate at 600 cells, 160 μL/well. After incubating overnight, compound solutions of gradient concentration was prepared, and added 40 μL of DMSO solution of the test compound of each concentration to the cells of each well. The final concentration of the compound is 10000, 2000, 400, 80, 16, 3.2, 0.64, 0.12, 0.025, 0 nM (the final concentration of DMSO is 0.25%). Incubated for 96 h at 37° C. and 5% CO₂. 50 μL of Cell-titer Glo working solution was added to each well, shaked and mixed, and incubated at room temperature for 10 minutes. The multi-functional microplate reader read the luminescence value, and converted the luminescence value reading into inhibition percentage.

Inhibition percentage=(maximum value−reading)/(maximum value−minimum value)*100.

The “maximum value” was the DMSO control, the “minimum value” indicates the cell-free control group.

Graphpad Prism software was used to perform curve fitting and obtain IC₅₀ value.

Refer to Table 2 for the IC₅₀ data of some compounds in the examples inhibiting MIA-PACA-2 cells.

TABLE 2

| Example | IC₅₀ (nM) |
|---|---|
| 1 | B |
| 2 | A |
| 3 | A |
| 4 | C |
| 5 | B |
| 6 | A |
| 7 | C |
| 8 | B |
| 9 | B |
| 10 | C |
| 11 | C |
| 12 | C |
| 13 | A |
| 14 | B |
| 15 | C |
| 16 | B |
| 17 | A |
| 18 | C |

Among them, A means IC₅₀≤1 uM, B means 1 uM≤IC₅₀≤10 uM, C means IC₅₀>10 uM, the same below.

### Example 2: Cell Proliferation Assay

Pave the H358 cells on a 96-well ultra-low adsorption plate with 2000 cells and 190 μL/well. After incubating overnight, compound solutions of gradient concentration was prepared, and added 10 μL of DMSO solution of the test compound of each concentration to the cells of each well. The final concentration of the compound is 10000, 3333.3, 1111.1, 370.4, 123.5, 41.2, 13.7, 4.6, 1.5, 0 nM (the final concentration of DMSO is 0.25%). Incubated for 96 h at 37° C. and 5% CO₂. 50 μL of Cell-titer Glo working solution was added to each well, shaked and mixed, and incubated at room temperature for 10 minutes. The multi-functional microplate reader read the luminescence value, and converted the luminescence value reading into inhibition percentage.

Inhibition percentage=(maximum value−reading)/(maximum value−minimum value)*100.

The “maximum value” was the DMSO control, the “minimum value” indicates the cell-free control group.

Graphpad Prism software was used to perform curve fitting and obtain IC₅₀ value.

Refer to table 3 for the IC₅₀ data of some compounds in the Examples for inhibiting H358 cells.

TABLE 3

| Example | IC₅₀ (nM) |
|---|---|
| 1 | B |
| 2 | A |
| 3 | A |
| 4 | C |
| 5 | B |
| 6 | B |
| 7 | C |
| 8 | B |

Although the present invention has been fully described through its embodiments, it is worth noting that various changes and modifications are obvious to those skilled in the art. Such changes and modifications should be included in the scope of the appended claims of the present invention.

1. A compound of the Formula (I) or a tautomer, pharmaceutically-acceptable salt, solvate, chelate, non-covalent complex or prodrug thereof, 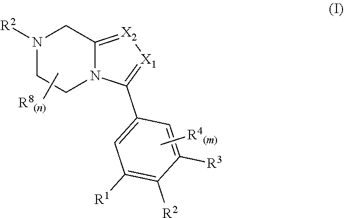
wherein,
R¹ or R³ is independently selected from the group consisting of H, amino, cyano, halogen, hydroxy, substituted or unsubstituted C₁₋₈ alkyl, substituted or unsubstituted C₃₋₈ cycloalkyl, substituted or unsubstituted C₂₋₈ alkene, and substituted or unsubstituted C₁₋₃ alkoxy;
R² is selected from the group consisting of substituted or unsubstituted C₃₋₁₀ cycloalkyl, substituted or unsubstituted C₃₋₁₀ heterocyclyl, substituted or unsubstituted C₆₋₁₂ aryl and substituted or unsubstituted C₅₋₁₂ heteroaryl;
R⁴ is selected from the group consisting of H, amino, cyano, halogen, hydroxy, substituted or unsubstituted C₁₋₈ alkyl, substituted or unsubstituted C₂₋₈ alkenyl, substituted or unsubstituted C₃₋₁₀ cycloalkyl, substituted or unsubstituted C₃₋₁₀ heterocyclyl, substituted or unsubstituted C₆₋₁₂ aryl, and substituted or unsubstituted C₅₋₁₂ heteroaryl; or
R⁴ and R¹ or together with R³ together with the atom to which it is attached form substituted or unsubstituted C₃₋₁₀ cycloalkyl, substituted or unsubstituted C₃₋₁₀ heterocyclyl, substituted or unsubstituted C₆₋₁₂ aryl, or substituted or unsubstituted C₅₋₁₂ heteroaryl;
X₁ is N or CR⁵, wherein, R⁵ is selected from the group consisting of H, amino, cyano, halogen, hydroxyl, substituted or unsubstituted C₁₋₈ alkyl, substituted or unsubstituted C₂₋₈ alkenyl, substituted or unsubstituted C₃₋₁₀ cycloalkyl, substituted or unsubstituted C₃₋₁₀ heterocyclyl, substituted or unsubstituted C₆₋₁₂ aryl, and substituted or unsubstituted C₅₋₁₂ heteroaryl;
X₂ is N or CR⁶, wherein, R⁶ is selected from the group consisting of H, amino, cyano, halogen, hydroxyl, substituted or unsubstituted C₁₋₈ alkyl, substituted or unsubstituted C₂₋₈ alkenyl, substituted or unsubstituted amido, substituted or unsubstituted aminopyridyl, and substituted or unsubstituted pyrrolidinyloxy;
R⁷ is substituted or unsubstituted acryloyl;
R⁸ is selected from the group consisting of H, C₁₋₈ alkyl, C₁₋₈ alkoxy and C₁₋₈ haloalkyl;
m or n are independently selected from the group consisting of 0, 1 and 2;
wherein any of heterocyclyl or heteroaryl optionally contains 1, 2 or 3 heteroatoms independently selected from the group consisting of N, O and S.

2. The compound as claimed in claim 1, wherein R¹ or R³ is independently selected from the group consisting of hydroxy, halogen, C₂₋₃ alkenyl, C₂₋₃ alkyl, cyclopropyl, C₁₋₃ alkoxy and haloalkyl substituted C₁₋₃ alkoxy.

3. The compound as claimed in claim 1, wherein R¹ or R³ is independently selected from the group consisting of halogen, C₂₋₃ alkenyl and —O(C₁₋₂ alkylene)CF₃.

4. The compound as claimed in claim 1, wherein R² is selected from the group consisting of C₃₋₁₀ cycloalkyl, C₃₋₁₀ heterocyclyl, C₆₋₁₂ aryl and C₅₋₁₂ heteroaryl, wherein any of the C₃₋₁₀ cycloalkyl, C₃₋₁₀ heterocyclyl, C₆₋₁₂ aryl or C₅₋₁₂ heteroaryl is unsubstituted or optionally substituted with halogen, hydroxy, amino or C₁₋₆ alkyl.

5. The compound as claimed in claim 1, wherein R² is 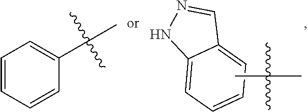
wherein 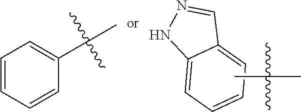
is optionally substituted by halogen, hydroxyl or amino.

6. The compound as claimed in claim 1, wherein R⁴ is selected from the group consisting of H, halogen and C₁₋₃ alkyl.

7. The compound as claimed in claim 1, wherein X₁ is N or CR⁵, wherein, R⁵ is selected from the group consisting of H, halogenated C₁₋₃ alkyl and 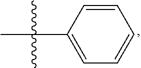 [SMILES: *c1ccccc1]
wherein 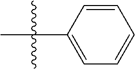 [SMILES: *c1ccccc1]
is unsubstituted or substituted by C₁₋₃ alkyl.

8. The compound as claimed in claim 1, wherein X₂ is N or CR⁶, wherein R⁶ is H.

9. The compound as claimed in claim 1, wherein R⁷ is halo or unsubstituted acryloyl.

10. The compound as claimed in claim 1, wherein R⁸ is selected from the group consisting of H, halogen and C₁₋₃ alkyl.

11. A compound, or a tautomer or pharmaceutically-acceptable salt thereof, wherein the compound is selected from the group consisting of:
1)1-(3-(2-Chloro-2′,6-difluoro-6′-hydroxy-[1,1′-biphenyl]-4-yl)-5,6-dihydro-[1,2,4]triazolo [4,3-a]pyrazin-7(8H)-yl)prop-2-en-1-one;
2)1-(3-(2,3′-Dichloro-6,6′-difluoro-2′-hydroxy-[1,1′-biphenyl]-4-yl)-5,6-dihydro-[1,2,4]triazolo[4,3-a]pyrazin-7(8H)-yl)prop-2-en-1-one; or
1-(3-(2,3′-Dichloro-2′,6-difluoro-6′-hydroxy-[1,1′-biphenyl]-4-yl)-5,6-dihydro-[1,2,4]triazolo[4,3-a]pyrazin-7(8H)-yl)prop-2-en-1-one;
3)1-(3-(2,3′,5′-Trichloro-2′,6-difluoro-6′-hydroxy-[1,1′-biphenyl]-4-yl)-5,6-dihydro-[1,2,4]Triazolo[4,3-a]pyrazin-7(8H)-yl)prop-2-en-1-one;
4)1-(3-(2-chloro-2′,6-difluoro-6′-hydroxy-[1,1′-biphenyl]-4-yl)-5,6-dihydro-[1,2,4]triazolo [4,3-a]pyrazin-7(8H)-yl)-2-fluoroprop-2-en-1-one;
5)1-(3-(3-chloro-5-fluoro-4-(5-methyl-1H-indazol-4-yl)phenyl)-5,6-dihydro-[1,2,4]triazolo [4,3-a]pyrazin-7(8H)-yl)prop-2-en-1-one;
6)1-(3-(2-Chloro-2′,6-difluoro-6′-hydroxy-[1,1′-biphenyl]-4-yl)-5,6-dihydroimidazo[1,2-a]pyrazin-7(8H)-yl)prop-2-en-1-one;
7)1-(3-(2′-Amino-2,3′-dichloro-6,6′-difluoro-[1,1′-biphenyl]-4-yl)-5,6-dihydro-[1,2,4]triazolo[4,3-a]pyrazin-7(8H)-yl)prop-2-en-1-one;
8)1-(3-(2-Chloro-2′,6-difluoro-6′-hydroxy-[1,1′-biphenyl]-4-yl)-5,6-dihydroimidazo[1,5-a]pyrazin-7(8H)-yl)prop-2-en-1-one;
9)1-(3-(2,3′-Dichloro-6,6′-difluoro-2′-hydroxy-[1,1′-biphenyl]-4-yl)-5,6-dihydroimidazo [1,5-a]pyrazin-7(8H)-yl)prop-2-en-1-one; or
1-(3-(2,3′-Dichloro-2′,6-difluoro-6′-hydroxy-[1,1′-biphenyl]-4-yl)-5,6-dihydroimidazo[1,5-a]pyrazin-7(8H)-yl)prop-2-en-1-one;
10)1-(3-(2,3′,5′-Trichloro-2′,6-difluoro-6′-hydroxy-[1,1′-biphenyl]-4-yl)-5,6-dihydroimidazo[1,5-a]pyrazin-7(8H)-yl)prop-2-en-1-one;
11)1-(3-(2,2′-Difluoro-6′-hydroxy-6-vinyl-[1,1′-biphenyl]-4-yl)-5,6-dihydro-[1,2,4] triazolo [4,3-a]pyrazin-7(8H)-yl)prop-2-en-1-one;
12)1-(3-(2-Chloro-2′,6-difluoro-6′-hydroxy-[1,1′-biphenyl]-4-yl)-2-(2-isopropylphenyl)-5,6-dihydroimidazo[1,2-a]pyrazin-7(8H)-yl)prop-2-en-1-one;
13) 1-(3-(2,3′-dichloro-6,6′-difluoro-2′-hydroxy-[1,1′-biphenyl]-4-yl)-2-(2-isopropylphenyl)-5,6-dihydroimidazo[1,2-a]pyrazin-7(8H)-yl)prop-2-en-1-one; or
1-(3-(2,3′-dichloro-2′,6-difluoro-6′-hydroxy-[1,1′-biphenyl]-4-yl)-2-(2-isopropylphenyl)-5,6-dihydroimidazo[1,2-a]pyrazin-7(8H)-yl)prop-2-en-1-one;
14)1-(2-(2-Isopropylphenyl)-3-(2,3′,5′-trichloro-2′,6-difluoro-6′-hydroxy-[1,1′-biphenyl]-4-yl)-5,6-dihydroimidazo[1,2-a]pyrazin-7(8H)-yl)prop-2-en-1-one;
15)1-(3-(2,3′-Dichloro-6,6′-difluoro-2′-hydroxy-[1,1′-biphenyl]-4-yl)-5,6-dihydroimidazo [1,2-a]pyrazin-7(8H)-yl)prop-2-en-1-one;
16) 1-(3-(2,3′-dichloro-6′-fluoro-2′-hydroxy-6-(2,2,2-trifluoroethoxy)-[1,1′-biphenyl]-4-yl)-5,6-dihydro-[1,2,4]triazolo[4,3-a]pyrazin-7(8H)-yl)prop-2-en-1-one;
17) 1-(3-(2-chloro-2′-fluoro-6′-hydroxy-6-(2,2,2-trifluoroethoxy)-[1,1′-biphenyl]-4-yl)-5,6-dihydro-[1,2,4]triazolo[4,3-a]pyrazin-7(8H)-yl)prop-2-en-1-one; and
18)1-(3-(2,3′-Dichloro-6,6′-difluoro-2′-hydroxy-[1,1′-biphenyl]-4-yl)-2-(trifluoromethyl)-5,6-dihydroimidazo[1,2-a]pyrazin-7(8H)-yl)prop-2-en-1-one.

12. A pharmaceutical composition comprising a therapeutically effective amount of at least one compound of claim 1 and at least one pharmaceutically acceptable carrier.

13. (canceled)

14. A method for treating a disease mediated by KRAS G12C comprising administering the compound of claim 1 and/or a pharmaceutical composition comprising a therapeutically effective amount of at least one compound of claim 1 and at least one pharmaceutically acceptable carrier to a subject.

15. The method of claim 14, wherein the disease mediated by KRAS G12C is a cancer.

16. The method of claim 15, wherein the cancer is selected from the group consisting of breast cancer, multiple myeloma, bladder cancer, endometrial cancer, gastric cancer, cervical cancer, rhabdomyosarcoma, non-small cell lung cancer, small cell lung cancer, pleomorphic lung cancer, ovarian cancer, esophagus cancer, melanoma, colorectal cancer, hepatocellular carcinoma, head and neck tumor, hepatobiliary cell carcinoma, myelodysplastic syndrome, malignant glioma, prostate cancer, thyroid cancer, xuwang's cell tumor, lung squamous cell carcinoma, lichenoid keratosis, synovial sarcoma, skin cancer, pancreatic cancer, testicular cancer and liposarcoma.
